(19) United States
(12) Patent Application Publication
(10) Pub. No.: US 2024/0216289 A1
(43) Pub. Date: Jul. 4, 2024

(54) LIPIDOID NANOPARTICLES FOR THE TREATMENT OF DISEASES AND DISORDERS

(71) Applicant: Trustees of Tufts College, Medford, MA (US)

(72) Inventors: Qiaobing Xu, Lexington, MA (US); Jinjin Chen, Medford, MA (US); Zhongfeng Ye, Medford, MA (US)

(21) Appl. No.: 18/288,484

(22) PCT Filed: May 2, 2022
(86) PCT No.: PCT/US22/27256

§ 371 Date: Oct. 26, 2023

Related U.S. Application Data
(60) Provisional application No. 63/182,245, filed on Apr. 30, 2021.

(51) Int. Cl. A61K 9/51; A61K 9/00; A61K 31/704; A61K 31/7105; A61K 39/00; A61K 39/12

(52) U.S. Cl. CPC A61K 9/5123; A61K 9/0019; A61K 31/704; A61K 31/7105; A61K 39/12; A61K 2039/53; A61K 2039/55516; A61K 2039/55555; A61K 2039/55561

(57) ABSTRACT

Disclosed are lipidoid compositions that are capable of treating or preventing certain diseases (e.g., cancer or viral infections). Also disclosed are pharmaceutical compositions, comprising the lipidoid compositions. The disclosure also relates to methods of using the lipidoid compositions, and related kits comprising the lipidoid compositions.

### RELATED APPLICATIONS

This application claims the benefit of priority to U.S. Provisional Patent Application 63/182,245, filed Apr. 30, 2021, the contents of which is fully incorporated by reference herein.

### BACKGROUND

The rapid-spreading global pandemic of COVID-19, caused by SARS-COV2, is inflicting enormous suffering and economical loss around the world. Like many other major infectious diseases in the human history, the cost-effective, sustained control of COVID-19 infection will depend on effective vaccines. According to WHO, over sixty vaccine candidates are currently being developed, utilizing diverse platforms such DNA, RNA, protein, viral vector and inactivated virus.

Effective coronavirus vaccines likely require induction of both neutralizing antibodies and cytotoxic CD8 T cell (CTL) responses. Compared to protein and inactivated virus, which primarily induce antibody responses, RNA, DNA and viral vector-based vaccines induce both antibody and cell-mediated immunity, because antigenic proteins are expressed in situ. mRNA is rapidly emerging as a powerful vaccine platform for infectious diseases because mRNA-based vaccines are superior as compared to viral vector-based vaccines, which are complicated by anti-vector immunity and practical issues of manufacturing feasibility, and DNA-based vaccines, which usually lack potency in humans. In addition, mRNA-based vaccines are built on precise but adaptable antigen design and have been shown to be well tolerated, immunogenic, and scalable. To date, an mRNA-based vaccine for COVID-19 has already entered phase I clinical trial. Despite the enormous promise, the success of mRNA-based and other coronavirus vaccine remains to be determined. Multiple approaches should be pursued in parallel in order to develop an effective vaccine as soon as possible.

For vaccine application, mRNA is usually encapsulated in lipid nanoparticles (LNP), and administered either intramuscularly (i.m.) or intradermally (i.d.), where cells at the injection site uptake mRNA-LNP and express the encoded protein. mRNA and LNP also exhibit adjuvant properties by activating innate immune responses through pathways such as RIG-I and MDA5. To induce CTL response, antigenic epitopes expressed by myocytes (in case of i.m. injection) and other cell types (in case of i.d. injection) must be cross-presented by dendritic cells (DC) to induce T cell response in draining lymph node (LN). Although DC are reported to take up mRNA and express the encoded protein following i.d. injection, it is not clear how efficient this process is. Direct delivery and expression of mRNA into DC in the draining LN would be expected to enable more efficient expression and presentation of antigenic epitopes by DC to induce CTL response and likely immune responses in general.

A potential challenge of coronavirus vaccine development is antibody-dependent enhancement (ADE), which occurs non-neutralizing and sub-neutralizing antibodies bind to the virus and enables Fc receptor mediated uptake of virus-antibody complex by phagocytic cells, such as macrophages, leading to efficient infection of these cells. ADE was first reported during natural infection of dengue virus and was recently documented following the suboptimal dengue vaccination. Therefore, an effective coronavirus vaccine must induce potent neutralizing antibody responses to minimize ADE.

The rapid spreading of SARS-COV-2 from December 2019 has led to more than 150 million COVID-19 cases all over the world, with more than 3 million deaths. The administration of SARS-COV-2 vaccines, including inactivated virus, viral vector, and mRNA, have brought great potential for the prevention of the infection and spread of the SARS-COV-2. Among all the approved vaccines, mRNA vaccine showed great advantage in the manufacturing and efficacy. However, some ‘breakthrough’ COVID-19 cases still occurred among people already vaccinated.

Furthermore, the preliminary success for the protection against infectious diseases inspired the development of cancer mRNA vaccine faced with more challenging. Compared with other traditional cancer vaccines, mRNA cancer vaccine showed great advantages. For example, mRNA cancer vaccine only results in the transient expression of tumor antigens, avoiding the possible mutation caused by DNA vaccine. Second, mRNA cancer vaccine can encode various antigens including full protein and peptide antigens in a similar process, showing the ability to integrate all required tumor antigens together. Finally, mRNA cancer vaccine can induce stronger humoral and cellular response than traditional inactivated pathogen or protein-based vaccines, which directly influence the therapeutic outcome. Owing to the superiority of caner mRNA vaccine, all leading companies expanded the application of mRNA vaccine to the field of cancer treatment, with more than twenty mRNA cancer vaccines in clinic trails.

mRNA and delivery system are two key factors, determining the efficacy of mRNA vaccine. First, the major limitation of the application of original mRNA is the high immunogenicity, which is overfilled until the modification of nuclear acids was discovered. Along with the addition of polyA tail and cap structure and HPLC purification, all these efforts make the in vivo application of mRNA to be possible. Second, the development of new generation of delivery systems, especially the lipid nanoparticle (LNP), significantly improved the stability and transfection efficacy of mRNA in human body. The developed LNPs for RNA delivery could divided in to three generation based on their function. In detail, the first generation are usually non-degradable, e.g., 1,2-Dioleoyl-3-dimethylaminopropane (DODAP) and 1,2-Dilinoleyloxy-N,N-dimethyl-3-aminopropane (DLin-DMA), showing modest transfection effect but concerning toxicity in vivo. The second generation with biodegradable linkers such as DLin-MC3-DMA effectively deliver small RNAs such as siRNA to liver, leading to high and durable knockdown of targeted serum proteins. The third generation including ALC-0315 and SM-102 exhibited high transfection effect of long chain mRNA in vivo, overfilling the design of COVID-19 mRNA vaccine.

Despite the rapid development of LNPs brings revolution for mRNA delivery, most of the reported LNPs applied in vivo always show very strong mRNA expression in liver, which is a double blade sword for their biomedical application. On the one hand, the high mRNA expression in liver benefits the treatment of diseases related to liver. On the other hand, for some application in other organs, the undesired transfection of mRNA in liver remains a threat to human body. Therefore, though the efficacy of mRNA delivered by LNPs reached to the high level especially in liver, the targeting expression of mRNA in vivo according to the application might be the key point of the next generation of LNPs. For mRNA cancer vaccine, the targeted delivery and expression of mRNA encoding tumor antigens in lymphoid organs might be a promising strategy to improve the efficacy and reduce the side effect of mRNA vaccines. Though many nanosystems can deliver cargo to lymphoid organs by modification of active-targeting ligands, the successful mRNA delivery and transfection were still seldom reported owing to the hard transfection of immune cells in vivo.

In view of the foregoing, there is an ongoing need to develop a new formulation of mRNA vaccines with higher efficacy for the treatment of certain diseases (e.g., viral infections and cancer).

### SUMMARY OF THE INVENTION

In one aspect, the present disclosure provides a nanoparticle, comprising a plurality of lipidoids; and an adjuvant, an antigen, or a nucleic acid, wherein each lipidoid has a structure represented by formula I:

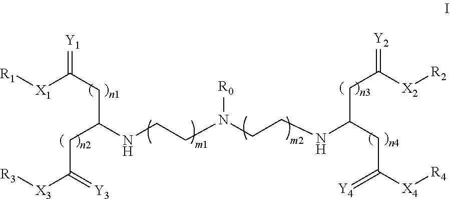

or a pharmaceutically acceptable salt thereof,
wherein,
each Y₁, Y₂, Y₃, Y₄, X₁, X₂, X₃, and X₄ is independently selected from the group consisting of O, S, and NR₅;
R₀ is H or alkyl;
R₁, R₂, R₃, and R₄ are each independently selected from the group consisting of alkyl, alkyloxyalkyl, alkylaminoalkyl, alkylthioalkyl, alklydisulphidealkyl, alkylamidoalkyl, alkylesteralkyl, alkylcarbamatealkyl, alkylcarbonatealkyl, and alkylurealylalkyl;
each R₅ is independently selected from hydrogen, alkyl, or aralkyl;
n1, n2, n3, and n4 are each independently 1, 2, 3, 4, 5, 6, 7, 8, 9, or 10; and
m1 and m2 are each independently 1, 2, 3 4, or 5.

In another aspect, the present disclosure provides pharmaceutical compositions comprising the nanoparticles disclosed herein and a pharmaceutically acceptable excipient.

In another aspect, the present disclosure provides kits comprising the nanoparticles disclosed herein and a chemotherapeutic agent.

In yet another aspect, the present disclosure provides methods of treating or prevent certain diseases (e.g., cancer and viral infections) in a subject comprising administering the nanoparticles disclosed herein to the subject.

### BRIEF DESCRIPTION OF THE DRAWINGS

FIG. 1 shows the expression of S protein in the cell lysate and medium of HEK-293 cells after transfection with S or Sₚₚ mRNA.

FIG. 2A shows the Chemical structure of 113-O12B.

FIG. 2B shows the optimization of 113-O12B lipids for enhanced lymph node delivery of Luc mRNA.

FIG. 3 shows the percentage of tdtomato⁺ cells in CD11c, F4/80, CD3, and B220 positive cells.

FIG. 4A shows the antibody titer of IgG at day 28 after the vaccination.

FIG. 4B shows the antibody titer of IgG1 at day 28 after the vaccination.

FIG. 4C shows the antibody titer of IgG2a at day 28 after the vaccination.

FIG. 4D shows the ratio of IgG2a and IgG1 in the antibody titer.

FIG. 5A shows the pictures of ELISpot assays.

FIG. 5B shows the quantitative analysis of IFN γ from positive T cells in the ELISpot assays in FIG. 5A.

FIG. 6A shows an exemplary route to a library of biodegradable lipidoids.

FIG. 6B shows the exemplary chemical structures of amine heads and carbon tails for lipidoids library construction.

FIG. 6C shows in vivo lymph node targeted delivery of Luciferase mRNA using lipidoid 93-O17S after intradermal injection at the base of the tail in mice.

FIG. 7 shows the design of mRNA for vaccine design.

FIG. 8 shows that Pam3CSK4 formulated at the weight ratio of 2.5% decreased the mRNA expression in draining lymph nodes.

FIGS. 9A and 9B show that the addition of PAM3CSK4 increase IL-4+ T cell response.

FIGS. 10A-10C show the screening of top-performing 5′UTR in HEK293T cells using pcDNA3.0-eGFP. FIG. 10A is a schematic of fragments inserted into 5′ terminal of eGFP on pcDNA3.0-eGFP. FIG. 10B shows the expression level of each 5′UTR modified pcDNA3.0-eGFP in HEK293T cells at different time points (36 hrs, 60 hrs, 84 hrs and 108 hrs). FIG. 10C shows representative flow images and the quantitated percentages of eGFP+ HEK293T cell population at each time point (36 hrs, 60 hrs, 84 hrs and 108 hrs). The highlighted frames indicate the two best-performing 5′UTR candidates for pcDNA3.0-eGFP expression enhancement, 70nt and Ces1d.

FIGS. 11A-11C show the screening of top-performing 3′UTR in HEK293T cells using pcDNA3.0-Ces1d-eGFP. FIG. 11A shows the schematic of fragments inserted into the 3′ terminal of cGFP on pcDNA3.0-Ces1d-eGFP. FIG. 11B shows the expression level of 3′UTR modified pcDNA3.0-Ces1d-eGFP in HEK293T cells at each time point (48 hrs, 72 hrs, 96 hrs and 120 hrs). The dashed line indicated plasmids with different 3′UTR substitution reached to the peak of protein expression at 72 hrs and 96 hrs, respectively. FIG. 11C shows representative flow images and the quantitated percentages of eGFP+ HEK293T cell population with different pcDNA3.0-Ces1d-eGFP-3′UTR treatment at each time point (36 hrs, 60 hrs, 84 hrs and 108 hrs). The highlighted frames indicate the top-performing 3′UTR candidates for pcDNA3.0-Ces1d-eGFP expression.

FIGS. 12A-12D show the screening of top-performing 3′UTR in HEK293T cells using pcDNA3.0-70nt-eGFP. FIG. 12A shows the schematic of fragments inserted into the 3′ terminal of cGFP on pcDNA3.0-70nt-cGFP. FIGS. 12B and 12C show the expression level of 3′UTR modified pcDNA3.0-Ces1d-cGFP in HEK293T cell at each time point (48 hrs, 72 hrs, 96 hrs and 120 hrs). The dashed line indicates plasmids with different 3′UTR substitution reach the peak of protein expression at 72 hrs, 96 hrs and 120 hrs, respectively.

FIG. 12D shows representative flow images and the quantitated percentages of eGFP+ HEK293T cell population under different pcDNA3.0-Ces1d-eGFP-3′UTR treatment at each time point (36 hrs, 60 hrs, 84 hrs and 108 hrs). Red frame highlighted the top-performed 3′UTR candidates on pcDNA3.0-70nt-cGFP.

FIGS. 13A-13E show the optimization of lipid 88 LNP formulations using 5omU-Fluc mRNA. FIG. 13A shows the structure of lipidoid 88. FIG. 13B shows the preliminary screening of lipid 88 LNP formulations with helper lipids (e.g., Chol, DOPC, and DMG-PEG). FIG. 13C shows images of living mice injected with TriLink 5omU-Fluc mRNA loaded multiple lipid 88 formulations, and commercial lipid ALC0315. Images were taken by IVIS imaging system at 6 hr post IV injection. FIGS. 13D and 13E show the further screening of lipid 88 formulations with a weight ratio of 16:4:2:1 (L88:Chol:DOPC:DMG-PEG).

FIGS. 14A-14D show the screening of top-performing 3′UTR in mice liver using Cleancap AG-Ces1d-Fluc-cGFP mRNA format. FIG. 14A shows a schematic of Cleancap AG-Ces1d-Fluc-eGFP mRNA construct in vitro transcription. FIG. 14B shows that the quality of modified mRNA was validated on 1% agarose gel (RNase-free). FIG. 14C shows the efficacy of Ces1d and 3′UTRs on Cleancap AG-Ces1d-Fluc-eGFP mRNA expression and stability in mice liver. FIG. 14D shows imaging of mice injected with 3′UTR modified Ces1d-Fluc-eGFP mRNA loaded optimal lipid 88 LNP.

FIGS. 15A-15D show the screening of top-performing 3′UTR in mice liver using Cleancap AG-70nt-Fluc-eGFP mRNA format. FIG. 15A shows a schematic of Cleancap AG-70nt-Fluc-cGFP mRNA construct in vitro transcription. FIG. 15B shows that the quality of modified mRNA was validated on 1% agarose gel (RNase-free). FIG. 15C shows the efficacy of Ces1d and 3′UTRs on Cleancap AG-70nt-Fluc-cGFP mRNA expression and stability in mice liver. FIG. 15D shows the imaging of mice injected with 3′UTR modified 70nt-Fluc-cGFP mRNA loaded optimal Lipid 88 LNP.

FIGS. 16A and 16B show the luciferase expression of Fluc-cGFP mRNA with Cleancap AG capping or ARCA capping in mice liver delivery. CleanCap AG showed 3-fold higher translational efficacy than that of ARCA under the same mRNA construct, T7-α-globin-Fluc-eGFP-Mmβ-globin (System Biosciences, US).

FIGS. 17A-17G show Screening and optimization of LNPs with targeting ability of LNs. FIG. 17A shows the exemplary chemical structure of lipidoids. FIG. 17B shows the bioluminescence within inguinal LNs after treatment with LNP/mLuc subcutaneously at tail base for 6 h. FIG. 17C shows the bioluminescence within inguinal LNs after treatment by LNP/mLuc with different formulations. FIG. 17D shows typical images of bioluminescence distribution of mice. FIG. 17E shows the bioluminescence within inguinal LNs after treatment by LNP/mLuc with different formulations. FIG. 17F illustrates the mechanism of subcellular analysis of mRNA expression in Ai14 reporter mice. FIG. 17G shows the percentage of tdTomato positive cells in different type of immunocytes after treatment with LNP/mCre subcutaneously at tail base for 48 h.

FIGS. 18A-18G show the exemplary T cell response and protection effect by vaccination with cancer mRNA vaccine. FIG. 18A illustrates an exemplary timeline for vaccination and bleeding. FIGS. 18B and 18C show the changes of cytokines and chemokines to untreated mice after 24 of treatment by blank or OVA mRNA formulated LNPs. FIG. 18D show exemplary flow cytometry images of IFN-γ positive cells within CD3⁺CD8⁺ T cells after 1 week of 2^{nd} vaccination. FIG. 18E shows the time-dependent changes of IFN-γ positive cells after 7, 14, and 28 days of 2^{nd} vaccination. FIG. 18F shows exemplary tumor volumes of B16F10-OVA xenograft tumor model. FIG. 18G shows an image of the lungs after 18 days of rechallenging by B16F10-OVA cell intravenously.

FIGS. 19A-19F show the changes of the immunocellular composition in established B16F10-OVA tumor after vaccination. FIG. 19A illustrates an timeline for tumor inoculation and vaccination. FIGS. 19B and 19C show the changes of T cells and APCs within tumor after 7 days of 2^{nd} vaccination. FIG. 19D shows exemplary flow cytometry images and percentages of FoxP⁺ cells within CD3⁺ CD4⁺ T cells after the vaccination. FIG. 19E shows exemplary flow cytometry images of images of CD86+ cells within CD11b⁺ F4/80⁺ cells. FIG. 19F shows the Ratio of M1 to M2 macrophages after the vaccination.

FIGS. 20A-20D show the therapeutic effect on B16F10-OVA tumor after vaccination by cancer mRNA vaccine. FIG. 20A shows exemplary flow cytometry images and percentages of OVA peptide (SIINFEKL)-specific cells within CD3⁺ CD8⁺ T cells in spleen after 7 days of 2^{nd} vaccination. FIG. 20B shows ELISpot images and spot numbers of INF-γ secreting T cells in spleen of vaccinated mice. FIG. 20C shows the tumor volumes of B16F10-OVA xenograft model during the experiments. FIG. 20D shows lungs collected on 18 days from the mice with or without treatment. The treated mice showed a complete response.

FIGS. 21A-21D show the therapeutic effect on original B16F10 tumor after vaccination by cancer mRNA vaccine. FIG. 21A shows an IVT of TRP2₁₈₀-188 mRNA. FIG. 21B shows exemplary flow cytometry images and percentage of IFN-γ positive cells within CD3⁺ CD8⁺ T cells after 7 days of 2nd vaccination. FIG. 21C shows the tumor volumes of B16F10 xenograft model during the experiments. FIG. 21D shows lungs collected on 18 days from the mice with survived during the therapy or without treatment.

FIG. 22 shows the pKa and size of exemplary LNPs

FIGS. 23A and 23B show exemplary flow cytometry pictures for analysis of tdTomato positive cells with LNs after treatment with LNP/mCre.

FIG. 24 shows gating information for flow results of intracellular cytokine staining.

FIGS. 25A and 25B show gating information for an immunocellular composition experiment.

### DETAILED DESCRIPTION OF THE INVENTION

In one aspect, disclosed herein are serials LNPs with inherent targeting ability to liver, spleen, and lung without additional modification of targeting ligands. For example, a LNP with inherent lymph node (LN) targeting ability named 113-O12B was explored and applied for therapeutic cancer mRNA vaccine. Compared with the clinic LNP ALC-0315, 113-O12B showed significantly reduced mRNA expression in liver but higher expression in LNs after subcutaneous injection. The targeting delivery of ovalbumin (OVA)-approved encoding mRNA vaccine showed excellent CD8⁺ T cell response and exhibited excellent protection effect against OVA-transduced B16F10 cells. Moreover, vaccination with 113-O12B formulated with mRNA encoding model OVA protein antigen or tumor associated peptide antigen (TR_{P2180-188}) both achieved great therapeutic effect to established tumor models. Notably, the combination with anti-PD-1 further improved the complete response to these established tumor models. More importantly, all the mice surviving from the therapeutic experiment resisted the challenging of metastatic model, revealing long-term anti-tumor immunity generated by our cancer mRNA vaccine.

Furthermore 113-O12B showed a targeted delivery of mRNA to lymph node and exhibited the highest transfection of antigen presenting cells (macrophage and DC). After vaccination, 113-F2 showed similar antibody response compared with ALC-0315, while 113-F2 and 113 F2+PAM exhibited increased T cells, which could benefit the protection of infection by SARS-COV-2 by cellular immunity.

In one aspect, the present disclosure provides a nanoparticle, comprising a plurality of lipidoids; and an adjuvant, an antigen, or a nucleic acid, wherein each lipidoid has a structure represented by formula I:

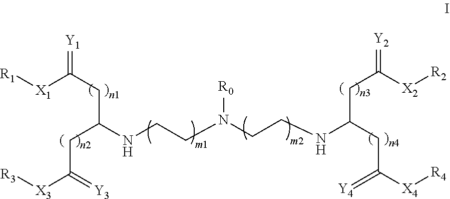

or a pharmaceutically acceptable salt thereof,
wherein,
each Y₁, Y₂, Y₃, Y₄, X₁, X₂, X₃, and X₄ is independently selected from the group consisting of O, S, and NR₅;
R₀ is H or alkyl;
R₁, R₂, R₃, and R₄ are each independently selected from the group consisting of alkyl, alkyloxyalkyl, alkylaminoalkyl, alkylthioalkyl, alklydisulphidealkyl, alkylamidoalkyl, alkylesteralkyl, alkylcarbamatealkyl, alkylcarbonatealkyl, and alkylurealylalkyl;
each R₅ is independently selected from hydrogen, alkyl, or aralkyl;
n1, n2, n3, and n4 are each independently 1, 2, 3, 4, 5, 6, 7, 8, 9, or 10; and
m1 and m2 are each independently 1, 2, 3 4, or 5.

In certain embodiments, each lipidoid has structure represented by formula I.

In certain embodiments, R₀ is methyl or ethyl. In certain embodiments, R₀ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide. In certain embodiments, R₀ is substituted with hydroxyl.

In certain embodiments, Y₁ is O.

In certain embodiments, Y₂ is O.

In certain embodiments, Y₃ is O.

In certain embodiments, Y₄ is O.

In certain embodiments, X₁ is O. In certain embodiments, X₁ is NH.

In certain embodiments, X₂ is O. In certain embodiments, X₂ is NH.

In certain embodiments, X₃ is O. In certain embodiments, X₃ is NH.

In certain embodiments, X₄ is O. In certain embodiments, X₄ is NH.

In certain embodiments, n1 is 2.

In certain embodiments, n2 is 2.

In certain embodiments, n3 is 2.

In certain embodiments, n4 is 2.

In certain embodiments, m1 is 1.

In certain embodiments, m2 is 1.

In certain embodiments, the lipidoid has a structure represented by formula Ia:

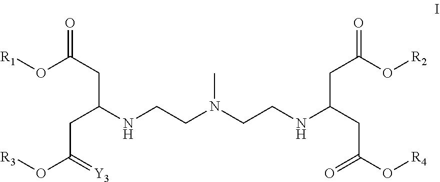

or a pharmaceutically acceptable salt thereof.

In certain embodiments, R₁ is alkyloxyalky. In certain embodiments, R₁ is alkylthioalkyl. In certain embodiments, R₁ is alklydisulphidealkyl. In certain embodiments, R₁ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

In certain embodiments, R₂ is alkyloxyalky. In certain embodiments, R₂ is alkylthioalkyl. In certain embodiments, R₂ is alklydisulphidealkyl. In certain embodiments, R₂ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

In certain embodiments, R₃ is alkyloxyalky. In certain embodiments, R₃ is alkylthioalkyl. In certain embodiments, R₃ is alklydisulphidealkyl. In certain embodiments, R₃ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

In certain embodiments, R₄ is alkyloxyalky. In certain embodiments, R₄ is alkylthioalkyl. In certain embodiments, R₄ is alklydisulphidealkyl. In certain embodiments, R₄ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

In certain embodiments, R₁ has a structure represented by formula IIa:

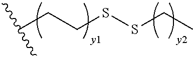

wherein,
y1 is 1, 2, 3, 4 or 5;
y2 is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
the wavy line indicates a connection to X₁.

In certain embodiments y1 is 1.

In certain embodiments, y2 is 5. In certain embodiments, y2 is 7. In certain embodiments, y2 is 9. In certain embodiments, y2 is 11. In certain embodiments, y2 is 13.

In certain embodiments, R₂ has a structure represented by formula IIb:

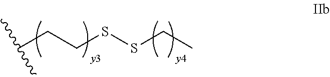

wherein,
y3 is 1, 2, 3, 4 or 5;
y4 is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
the wavy line indicates a connection to X₂.

In certain embodiments y3 is 1.

In certain embodiments, y4 is 5. In certain embodiments, y4 is 7. In certain embodiments, y4 is 9. In certain embodiments, y4 is 11. In certain embodiments, y4 is 13.

In certain embodiments, R₃ has a structure represented by formula IIc:

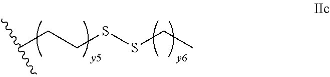

wherein,
y5 is 1, 2, 3, 4 or 5;
y6 is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
the wavy line indicates a connection to X₃.

In certain embodiments y5 is 1.

In certain embodiments, y6 is 5. In certain embodiments, y6 is 7. In certain embodiments, y6 is 9. In certain embodiments, y6 is 11. In certain embodiments, y6 is 13.

In certain embodiments, R₄ has a structure represented by formula IId:

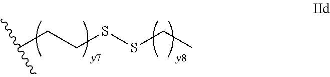

wherein,
y7 is 1, 2, 3, 4 or 5;
y8 is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
the wavy line indicates a connection to X₄.

In certain embodiments y7 is 1.

In certain embodiments, y8 is 5. In certain embodiments, y8 is 7. In certain embodiments, y8 is 9. In certain embodiments, y8 is 11. In certain embodiments, y8 is 13.

In certain embodiments, the plurality of lipidoids forms a bilayer.

In certain embodiments, the nanoparticle comprises an adjuvant. In certain embodiments, the adjuvant is a stimulator of the immune system. In certain embodiments, the stimulator of the immune system stimulates innate immunity. In certain embodiments, the stimulator of the immune system is a stimulator of interferon genes (STING). In certain embodiments, the STING is a STING agonist. In certain embodiments, the STING agonist is a cyclic dinucleotide. In certain embodiments, the STING agonist is cyclic guanosine monophosphate-adenosine monophosphate (cGAMP). In certain embodiments, the adjuvant is encapsulated within the nanoparticle. In certain embodiments, the plurality of lipidoids forms a bilayer and the adjuvant is encapsulated within the bilayer.

In certain embodiments, the nanoparticle comprises an antigen. In certain embodiments, the antigen is a vaccine. In certain embodiments, the antigen is a protein. In certain embodiments, the antigen is an attenuated virus. In certain embodiments, the antigen is encapsulated within the nanoparticle. In certain embodiments, the plurality of lipidoids forms a bilayer and the antigen is encapsulated within the bilayer.

In certain embodiments, the nanoparticle comprises a nucleic acid. In certain embodiments, the nucleic acid is a DNA or a RNA. In certain embodiments, the nucleic acid is an RNA. In certain embodiments, the RNA is an mRNA. In certain embodiments, when the mRNA contacts a cell, the mRNA induces the synthesis of a protein belonging to a cancer cell. In certain embodiments, the cancer cell is a bladder cancer cell, breast cancer cell, brain cancer cell, bone cancer cell, cervical cancer cell, colorectal cancer cell, head cancer cell, neck cancer cell, kidney cancer cell, liver cancer cell, lung cancer cell, lymphoma cell, mesothelioma cell, myeloma cell, prostate cancer cell, skin cancer cell, thyroid cancer cell, ovarian cancer cell, or uterine cancer cell. In certain embodiments, the mRNA contacts a cell, the mRNA induces the synthesis of a protein belonging to a virus. In certain embodiments, the virus is hepatitis C, norovirus, junin, dengue virus, coronavirus, human immunodeficiency virus, herpes simplex, avian flu, chickenpox, cold sores, common cold, glandular fever, influenza, measles, mumps, pharyngitis, pneumonia, rubella, severe acute respiratory syndrome, and lower or upper respiratory tract infection (e.g., respiratory syncytial virus). In certain embodiments, the virus is an influenza virus. In certain embodiments, the virus is a human immunodeficiency virus. In certain embodiments, the virus is a coronavirus. In certain embodiments, the coronavirus is SARS-CoV-2. In certain embodiments, the SARS-COV-2 is the alpha, beta, gamma, delta, omicron, or BA.2 strain of SARS-COV-2. In certain embodiments, when the mRNA contacts a cell, the mRNA induces the synthesis of the spike protein of the SARS-COV-2. In certain embodiments, the nucleic acid has at least 60%, 65%, 70%, 75%, 80%, 85%, 90%, 95%, or 99% sequence identity to any one of the sequences recited in Sequences—Table 1, Sequences—Table 2, or Sequences—Table 3. In certain embodiments, the nucleic acid has at least 75%, 80%, 85%, 90%, 95%, or 99% sequence identity to any one of the sequences recited in Sequences—Table 1, Sequences—Table 2, or Sequences—Table 3. In certain embodiments, the nucleic acid has at least 90%, 95%, or 99% sequence identity to any one of the sequences recited in Sequences—Table 1, Sequences—Table 2, or Sequences—Table 3. In certain embodiments, the nucleic acid has at least 95%, or 99% sequence identity to any one of the sequences recited in Sequences—Table 1, Sequences—Table 2, or Sequences—Table 3. In certain embodiments, the nucleic acid has a sequence according to any one of the sequences recited in Sequences—Table 1, Sequences—Table 2, or Sequences—Table 3. In certain embodiments, the nucleic acid is encapsulated within the nanoparticle. In certain embodiments, the plurality of lipidoids forms a bilayer and the nucleic acid is encapsulated within the bilayer.

In certain embodiments, the nanoparticle further comprises a chemotherapeutic agent. In certain embodiments, the chemotherapeutic agent is cytotoxic. In certain embodiments, the chemotherapeutic agent is an alkylating agent, an antimetabolite, an anti-microtubule agent, anti-tumor anti-biotic, or a topoisomerase inhibitor, a mitotic inhibitor, or a corticosteroid. In certain embodiments, the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat. In certain embodiments, the chemotherapeutic agent is doxorubicin. In certain embodiments, the chemotherapeutic agent is encapsulated within the nanoparticle. In certain embodiments, the nanoparticles are capable of internalizing an antigen (e.g., an antigen from a cancer cell).

In certain embodiments, the nanoparticle has a diameter of 25-500 nm. In certain embodiments, the nanoparticle has a diameter of 50-250 nm. In certain embodiments, the nanoparticle has a diameter of about 50 nm, about 75 nm, about 100 nm, about 125 nm, about 150 nm, about 175 nm, about 200 nm, about 225 nm, or about 250 nm. In certain embodiments, the nanoparticle has a diameter of about 50 nm, about 75 nm, about 100 nm, about 125 nm, about 150 nm, about 175 nm, or about 200 nm.

In certain embodiments, the nanoparticle has a pKa of 7-8. In certain embodiments, the nanoparticle has a pKa of about 7.0, about 7.1, about 7.2, about 7.3, about 7.4, about 7.5, about 7.6, about 7.7, about 7.8, about 7.9, or about 8.0. In certain embodiments, the nanoparticle has a pKa of about 7.3, about 7.4, about 7.5, about 7.6, or about 7.7.

In another aspect, the present disclosure provides pharmaceutical compositions comprising the nanoparticles disclosed herein and a pharmaceutically acceptable excipient.

In yet another aspect, the present disclosure provides methods of treating cancer in a subject in need thereof comprising administering a therapeutically effective amount of a nanoparticle disclosed herein or a pharmaceutically acceptable salt thereof to the subject.

In yet another aspect, the present disclosure provides methods of treating cancer in a subject in need thereof comprising the steps of administering a therapeutically effective amount of a chemotherapeutic agent to the subject; and
administering a therapeutically effective amount of a nanoparticle disclosed herein or a pharmaceutically acceptable salt thereof to the subject.

In certain embodiments, the chemotherapeutic agent is cytotoxic. In certain embodiments, the chemotherapeutic agent is an alkylating agent, an antimetabolite, an anti-microtubule agent, anti-tumor antibiotic, or a topoisomerase inhibitor, a mitotic inhibitor, or a corticosteroid. In certain embodiments, the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat. In certain embodiments, the chemotherapeutic agent is doxorubicin.

In certain embodiments, the nanoparticle is administered intratumorally.

In certain embodiments, the chemotherapeutic is administered intratumorally.

In certain embodiments, the nanoparticle is administered about 6-48 hours after the chemotherapeutic agent. In certain embodiments, the nanoparticle is administered about 6-24 hours after the chemotherapeutic agent. In certain embodiments, the nanoparticle is administered about 24 hours after the chemotherapeutic agent.

In certain embodiments, the cancer is a solid tumor. In certain embodiments, the cancer is bladder cancer, breast cancer, brain cancer, bone cancer, cervical cancer, colorectal cancer, head cancer, neck cancer, kidney cancer, liver cancer, lung cancer, lymphoma, mesothelioma, myeloma, prostate cancer, skin cancer, thyroid cancer, ovarian cancer, or uterine cancer.

In certain embodiments, the method elicits an anti-cancer immune response in the subject.

In certain embodiments, the method vaccinates the subject against the cancer.

In yet another aspect, the present disclosure provides methods of treating a viral infection in a subject in need thereof comprising administering a therapeutically effective amount of a nanoparticle disclosed herein or a pharmaceutically acceptable salt thereof to the subject.

In certain embodiments, the methods treat the viral infection.

In certain embodiments, the methods prevent the viral infection.

In certain embodiments, the viral infection is hepatitis C, norovirus, junin, dengue virus, coronavirus, human immunodeficiency virus, herpes simplex, avian flu, chickenpox, cold sores, common cold, glandular fever, influenza, measles, mumps, pharyngitis, pneumonia, rubella, severe acute respiratory syndrome, and lower or upper respiratory tract infection (e.g., respiratory syncytial virus). In certain embodiments, the viral infection is an influenza virus. In certain embodiments, the viral infection is a human immunodeficiency virus. In certain embodiments, the viral infection is a coronavirus. In certain embodiments, the coronavirus is SARS-COV-2. In certain embodiments, the SARS-COV-2 is the alpha, beta, gamma, delta, omicron strain, or BA.2 of SARS-COV-2. In certain embodiments, the method elicits an immune response in the subject. In certain embodiments, the method elicits an antiviral immune response in the subject. In certain embodiments, the method vaccinates the subject against the viral infection.

In another aspect, the present disclosure provides kits comprising the nanoparticles disclosed herein and a chemotherapeutic agent.

In another aspect, the present disclosure provides kits comprising the pharmaceutical compositions disclosed herein and a chemotherapeutic agent.

In certain embodiments, the chemotherapeutic agent is cytotoxic. In certain embodiments, the chemotherapeutic agent is an alkylating agent, an antimetabolite, an anti-microtubule agent, anti-tumor antibiotic, or a topoisomerase inhibitor, a mitotic inhibitor, or a corticosteroid. In certain embodiments, the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat. In certain embodiments, the chemotherapeutic agent is doxorubicin.

### Pharmaceutical Compositions

The compositions and methods of the present invention may be utilized to treat an individual in need thereof. The pharmaceutical composition described herein may comprise a therapeutic or prophylactic composition, or any combination thereof. In certain embodiments, the lipidoid compositions may be assembled with an antigen, an immune modulator, or any combination thereof. In certain embodiments, the individual is a mammal such as a human, or a non-human mammal. When administered to an animal, such as a human, the composition or the lipidoid composition is preferably administered as a pharmaceutical composition comprising, for example, a lipidoid composition of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a lipidoid composition such as a lipidoid composition of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a lipidoid composition of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those lipidoid compositions, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The lipidoid composition may also be formulated for inhalation. In certain embodiments, a lipidoid composition may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the lipidoid composition which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active composition, such as a lipidoid (e.g., nanoparticle) composition as described herein, with the carrier and, optionally, one or more accessory ingredients. In general, the formulations are prepared by uniformly and intimately bringing into association a lipidoid (e.g., nanoparticle) composition as described herein with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a lipidoid (e.g., nanoparticle) composition as described herein of the present invention as an active ingredient. Lipidoid compositions may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium lipidoid compositions; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered lipidoid composition moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active lipidoid compositions, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active lipidoid composition may be mixed under sterile conditions with a pharmaceutically acceptable carrier, and with any preservatives, buffers, or propellants that may be required.

The ointments, pastes, creams and gels may contain, in addition to an active lipidoid composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active lipidoid composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a lipidoid composition of the present invention to the body. Such dosage forms can be made by dissolving or dispersing the active lipidoid composition in the proper medium. Absorption enhancers can also be used to increase the flux of the lipidoid composition across the skin. The rate of such flux can be controlled by either providing a rate controlling membrane or dispersing the lipidoid composition in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active lipidoid compositions in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject lipidoid compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active lipidoid compositions can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a lipidoid composition at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular lipidoid composition or combination of lipidoid compositions employed, or the ester, salt or amide thereof, the route of administration, the time of administration, the rate of excretion of the particular lipidoid composition(s) being employed, the duration of the treatment, other drugs, lipidoid compositions and/or materials used in combination with the particular lipidoid composition(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or lipidoid composition at levels lower than that required in order to achieve the desired therapeutic effect and gradually increase the dosage until the desired effect is achieved. By “therapeutically effective amount” is meant the concentration of a lipidoid composition that is sufficient to elicit the desired therapeutic effect. It is generally understood that the effective amount of the lipidoid composition will vary according to the weight, sex, age, and medical history of the subject. Other factors which influence the effective amount may include, but are not limited to, the severity of the patient's condition, the disorder being treated, the stability of the lipidoid composition, and, if desired, another type of therapeutic agent being administered with the lipidoid composition of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active lipidoid composition used in the compositions and methods of the invention will be that amount of the lipidoid composition that is the lowest dose effective to produce a therapeutic effect. Such an effective dose will generally depend upon the factors described above.

If desired, the effective daily dose of the active lipidoid composition may be administered as one, two, three, four, five, six or more sub-doses administered separately at appropriate intervals throughout the day, optionally, in unit dosage forms. In certain embodiments of the present invention, the active lipidoid composition may be administered two or three times daily. In preferred embodiments, the active lipidoid composition will be administered once daily.

The patient receiving this treatment is any animal in need, including primates, in particular humans; and other mammals such as equines, cattle, swine, sheep, cats, and dogs; poultry; and pets in general.

In certain embodiments, lipidoid compositions of the invention may be used alone or conjointly administered with another type of therapeutic agent.

The present disclosure includes the use of pharmaceutically acceptable salts of lipidoid compositions of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂-O-alkyl, —OP(O)(O-alkyl)₂ or —CH₂-OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

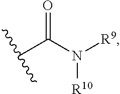

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

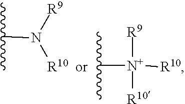

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

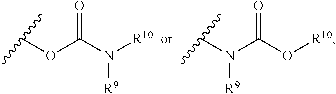

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

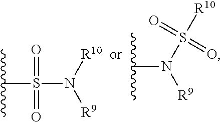

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

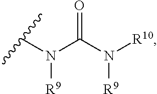

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds disclosed herein. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the lipidoid compositions (e.g., nanoparticles) useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Some of the lipidoid compositions (e.g., nanoparticles) may also comprise chemical compound which exist in tautomeric forms. Such forms, although not explicitly indicated in the formulae described herein, are intended to be included within the scope of the present disclosure.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

### EXAMPLES

The invention now being generally described, it will be more readily understood by reference to the following examples which are included merely for purposes of illustration of certain aspects and embodiments of the present invention, and are not intended to limit the invention.

### Example 1: Preparation of Exemplary Anti-Viral Nanoparticles

Production of Full S Protein mRNA

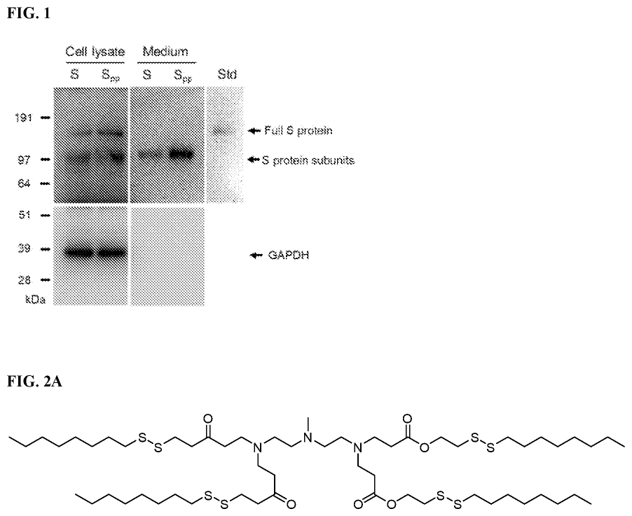

Two types of mRNA encoded for the full length of SARS-COV-2 spike protein (S protein) were developed, including wild type S and two proline (2P)-mutated (K986P and V987P) S (Sₚₚ) protein. The quality of mRNA was evaluated in HEK-293 cells in vitro. As shown in FIG. 1, the 2P mutation increased the expression of S proteins in cell lysate and culture medium, which is consistent with previous reported works.

### Optimization of Lipid Nanoparticles (LNPs)

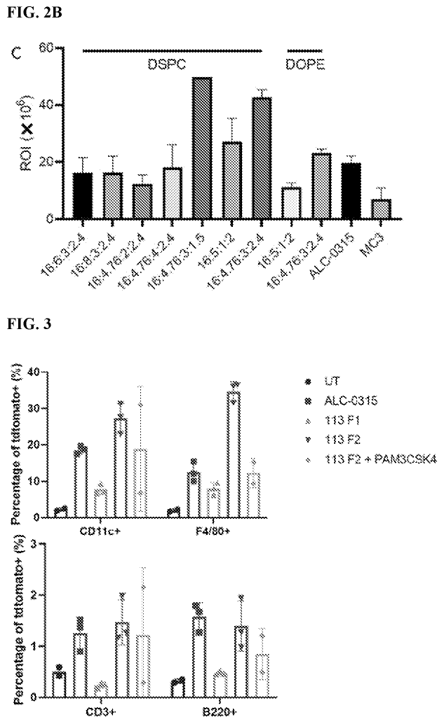

The LNPs showed great importance in the formulation of mRNA vaccines. From the library screening, it was discovered that the lipid 113-O12B (FIG. 2A) showed strong expression of luciferase (Luc) in the inguinal lymph nodes after subcutaneous injection of Luc mRNA at tail base of Balb/c mice. The formulation of 113 LNP was then further optimized (FIG. 3). The LNP was formulated using ionizable lipid (113-O12B), cholesterol (Chol), distearoylphosphatidylcholine (DSPC) or 1,2-dioleoyl-sn-glycero-3-phosphoethanolamine (DOPE), and 1,2-dimyristoyl-rac-glycero-3-methoxypolyethylene glycol-2000 (DMG-PEG) with different weight ratio. It was found that two formulations 113-F1 (16:4.76:3:1.5) and 113-F2 (16:4.76:3:2.4) showed better Luc expression in Inguinal lymph nodes than ALC-0315 (the lipid formulation used in Pfizer/biotech SARS-Cov2 vaccine (BNT162b2) formulation) and MC3 (lipid formulation used in FDA approved drug Onpattro). Additionally, ALC-0315 showed significantly increased expression of Luc in liver. 113-F1 and 113-F2 were chosen for the further immunization study.

### Identification of Subtypes of Transfected Cells in Inguinal Lymph Nodes

The types of cells in inguinal lymph nodes after s.c. delivery of Cre mRNA vaccines in Ai14 mice was further investigated. After 48 h of the s.c. injection of LNP/Cre mRNA complex, the inguinal lymph nodes were collected. The cell suspension was stained with marker of different immune cells, such as CD3 for T cells, B220 for B cells, F4/80 for macrophages, and CD11c for dendritic cells (DCs). The 113-F2 LNP showed highest transfection of macrophages and DCs, in which about 30% of cells were transfected (FIG. 3). The 113-F1 and ALC-0315 LNPs showed less efficacy compared with 113-F2 LNP. The addition of PAM3CSK4 (PAM), a TLR1/2 agonist, into the formulation decreased the transfection efficacy.

### Vaccination

The mRNA vaccine performance was evaluated using different LNP formulation in mice. Mice were immunized with 1 μg of mRNA in LNPs of ALC-0315, 113-F1, 113-F2, and 113-F2+PAM3CSK4 at day 1 and day 21. The blood and spleen were collected at day 28 for further analysis.

### Antibody Titer

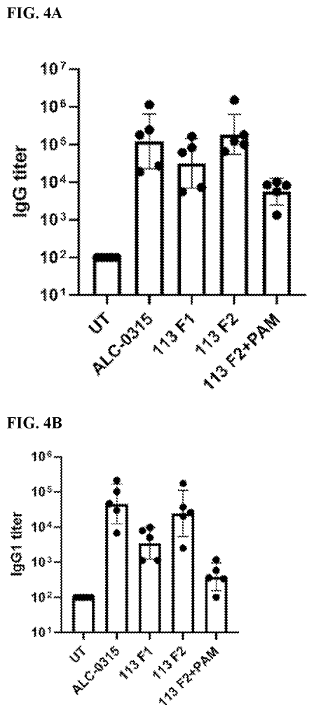

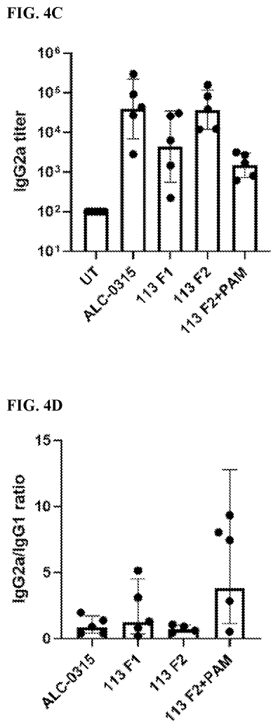

The antibody titers including IgG, IgG1, and IgG2a after vaccination were measured for the evaluation of antibody responses (FIGS. 4A-4D). The 113-F2 showed similar antibody titers compared with ALC-0315, while was higher than those of 113-F1 and 113-F2+PAM. However, the ratio of IgG2a to IgG1 in 113-F2+PAM was much higher than the other groups, indicating a stronger Th1 response than other groups.

### ELISpot Assay

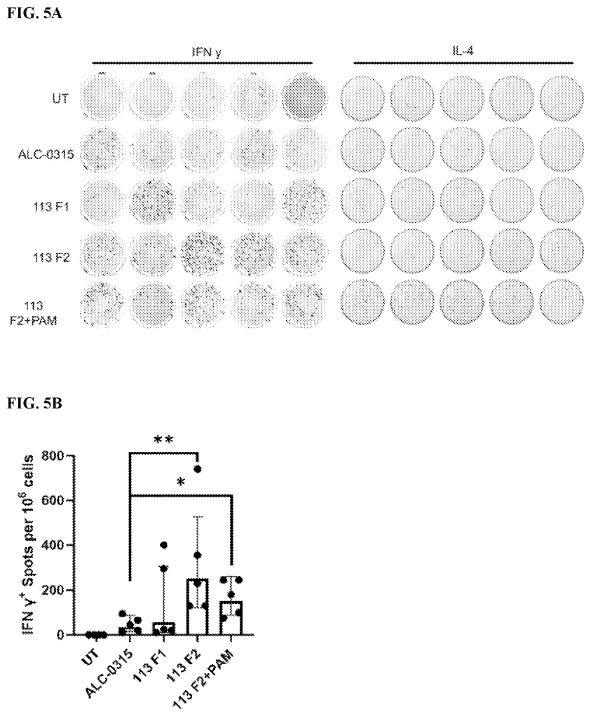

The T cell response after the vaccination was analyzed by IFN γ and IL-4 ELISpot (FIGS. 5A-C). The mice without vaccination showed almost no response of IFN γ and IL-4 positive T cells. The 113-F2 and 113-F2+PAM exhibited significantly higher response of IFN γ positive T cells than ALC-0315. However, only 113-F2+PAM showed increased number of IL-4 positive T cells compared with other groups.

To Enhance Immune Responses to Coronavirus mRNA Vaccine by Targeting mRNA to Dendritic Cells in Draining Lymph Nodes Through a Novel LNP.

The lipidoid library for mRNA delivery in vitro and in vivo was screened and lipidoids 93-O17S were identified to efficiently target mRNA cargo to draining lymph nodes following intradermal injection. The induction of CTL and antibody responses between lymph node-targeted mRNA delivery by 93-O17S and commonly used LNP in mice will be compared.

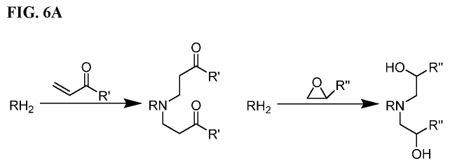

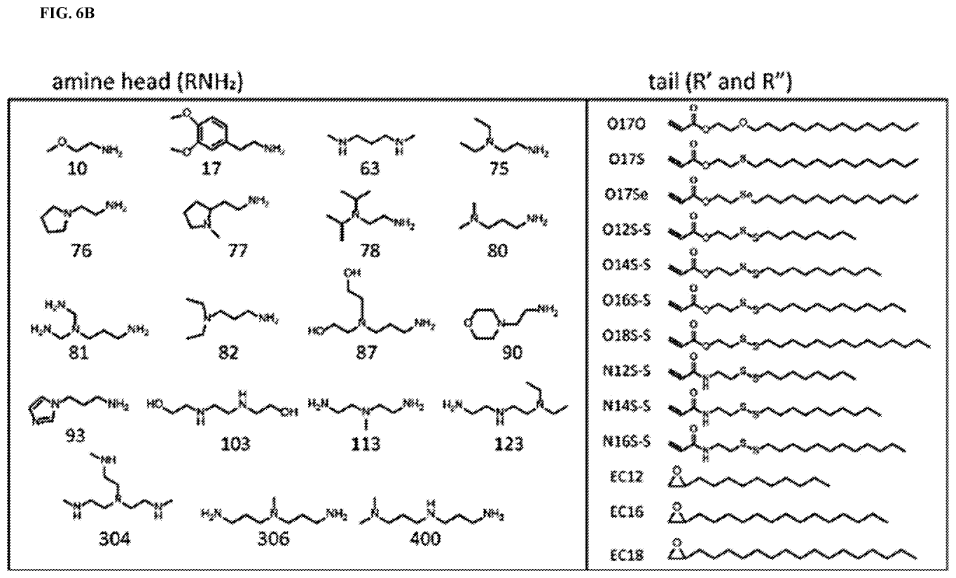

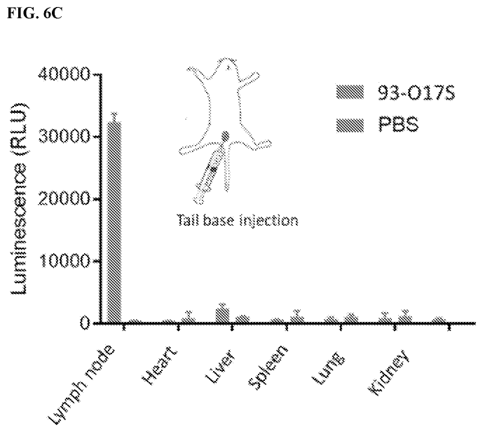

A library of biodegradable synthetic lipid molecules (247 lipidoids) by reacting amine head groups with long chain alkyl tail groups in a combinatorial fashion has been developed (FIGS. 6A & 6B) and screened for protein and nucleic acid delivery both in vitro and in vivo. A new class of imidazole containing lipidoids 93-O17S were identified that specifically deliver mRNA to T cells, macrophages and dendritic cells in the spleen following systemic injection, and to draining lymph nodes via intradermal injection (FIG. 6C). Therefore, 93-O17S is a promising LNP formulation for delivering antigen encoding mRNA to draining lymph nodes for vaccine application.

To determine the potency of 93-O17S encapsulated mRNA, mRNA encoding the secreted from of the COVID-19 S protein alone or S protein plus 2A and eGFP (or HA tag fused to the C-terminal of S protein) will be prepared and encapsulated in the standard LNP and 93-O17S LNP. First, to determine if 93-O17S stimulates mRNA delivery into DC in draining LN, mRNA-LNP (encoding both S protein and eGFP) will be injected i.d. at the base of the tail of C57BL/6 (B6) mice. PBS injected mice will serve as negative controls. Expression of eGFP by DC, macrophages, B cells, CD4+ and CD8+ T cells in draining LN will be analyzed by flow cytometry 2, 4, and 6 days after injection. Second, to determine if 93-O17S enhance immune responses, mRNA-LNP (encoding S protein alone) will be injected i.d. at the base of the tail of B6 mice at day 0 and day 28. PBS injected mice will serve as negative controls. Mice will be bled at day 7, 14, 21, 28 and 35 after the primary immunization to quantify the titer of IgG specific for S protein by ELISA and neutralizing antibody titer. Half of the mice per group will be sacrificed at day 21 and cells from spleen and draining LN will be stimulated with antigen presenting cells, which are prepared by transfecting bone marrow cells from non-immunized B6 mice with mRNA-LNP 24 hours earlier and then irradiated. 24 and 48 hours later, expression of IFN-g and TNFa by CD4 and CD8 T cells will be determined by flow cytometry and ELISPOT. The remaining mice will be sacrificed on day 35 and expression of IFN-g and TNF-α by CD4 and CD8 T cells will be determined as above. The experiments will be repeated with sufficient number of mice per group for statistical analysis. Results from these experiments will show 1) if 93-O17S LNP delivers mRNA to dendritic cells more effectively than standard LNP, 2) if 93-O17S LNP stimulates stronger T cell and antibody responses, and 3) if 93-O17S stimulates a stronger memory T and B cell responses. It is expected that LN-targeted delivery of mRNA likely induces more effective immune responses to coronavirus mRNA vaccine.

To Augment Neutralizing Antibody Responses by Using Toll-Like Receptor (TLR) Agonists that have been Shown to Enhance Neutralizing Antibody Responses.

It has been shown that a combination of TLR agonists Pam3CSK4 and poly I:C dramatically enhances neutralizing antibody responses to a protein-based dengue virus vaccine. Two TLR agonists will be tested individually or in combination to enhance neutralizing antibody responses to coronavirus mRNA vaccine.

### To Augment Neutralizing Antibody Responses by Using Toll-Like Receptor (TLR) Agonists.

Previously, it has been determined the combination of Pam3CSK4 and poly I:C augments neutralization antibody titer to dengue virus by ˜10-fold while reducing total reactive antibody titer by ˜10-fold as compared to alum. Pam3CSK4 and poly I:C individually and together will be tested to see if neutralizing antibody titer of coronavirus mRNA vaccine are enhanced. Pam3CSK4 or poly I:C or both will be encapsulated into mRNA-LNP (either standard LNP or 93-O17S, whichever is more potent, see aim #1) and used to immunized B6 mice i.d. on day 0 and day 28. Mice will be bled before immunization and 7, 14, 21, 28 and 35 days after primary immunization to measure total anti-S-protein IgG and neutralizing antibody titer following primary and secondary immunization. S-protein-specific T cell response will be analyzed as above following stimulation of cells from spleen and draining lymph nodes with irradiated S-protein expressing bone marrow antigen presenting cells as above. Results from these studies will show if incorporation of Pam3CSK4 and/or poly I:C into mRNA-LNP enhances neutralizing antibody titer as well as T cell responses. It is expected that Pam3CSK4 and/or poly I:C will enhance neutralizing antibody responses to mRNA vaccines.

Professor Ling Chen at Guangzhou Institute for Respiratory Health has conducted transcriptional analysis (RNAseq) of peripheral blood mononuclear cells (PBMCs) from COVID-19 infected patients at different days after infection. Transcription signatures in patient PBMCs will be analyzed and it will be determined if existing approved adjuvants and TLR agonists mimic the same transcriptional signatures. Additional new adjuvants or TLR agonists enhance neutralizing antibody responses to mRNA vaccine will also be investigated.

To Determine if the mRNA-LNP-Adjuvant Formulation Confer Protection Against COVID-19 Infection in ACE2 Transgenic Mice.

ACE2 transgenic mice will be immunized with the most potent formulation of mRAN-LNO-adjuvant as identified. Mice will be monitored for induction of CTL responses and neutralizing antibodies, and challenged with COVID-19 to determine the efficacy of protection.

To determine if the most potent mRNA-LNP-adjuvant formulation confers protection against COVID-19 infection in ACE2 transgenic mice. Once the most potent mRNA-LNP-adjuvant formation is identified with normal B6 mice, immunize ACE2 transgenic mice will be immunized with the most potent formulation. T cell responses will be validated at day 21 after primary immunization and 7 days after secondary immunization, and total anti-S-protein IgG titer and neutralizing antibody titer will be validated by ELISA and PRNT assay with sera at 7, 14, 21 and 28 days after primary immunization and 7 days after secondary immunization. Immunized mice will be challenged with COVID-19 28 days after primary or secondary immunization. Virus titer and the health status and survival of the mice will be monitored every 3 days. Results from this study will show if the combination of the lymph node-targeted delivery and TLR agonist adjuvant confer better protection against coronavirus challenge in mice.

In summary, the objectives ae to determine i) if LNP-targeting of mRNA, ii) if incorporation of specific adjuvants, and iii) the combination enhances both CTL and neutralizing antibody response to coronavirus mRNA vaccine and therefore confer better protection against COVID-19 infection. Positive results will contribute to the development of an effective mRNA vaccine for COVID-19. As the Pam3CSK4 and poly I:C are approved clinically, they can be readily incorporated into mRNA and probably protein-based vaccines.

The ionizable lipid 113-O12B showed a targeted delivery of mRNA to lymph node and exhibited the highest transfection of antigen presenting cells (macrophage and DC). After vaccination, 113-F2 showed similar antibody response compared with ALC-0315, while 113-F2 and 113 F2+PAM exhibited increased T cells, which could benefit the protection of infection by SARS-COV-2 by cellular immunity.

### SUMMARY

LNP formulations using ionizable lipid 113-O12B were identified for a targeted delivery of mRNA to lymph node. Higher transfection of antigen presenting cells (macrophage and DC) comparing with the LNP formulation using ionable lipid ALC-0315 was observed. ALC-0315 is the ionizable lipid used in SARS-Cov2 mRNA vaccine product from Pfizer/BioNTech. It was found 113 LNP formulation showed similar antibody response compared with ALC-0315 formulation, while 113 formulation with or without adjuvant (such as PAM3Cys) increases T cell response. The new formulation could benefit the protection of infection by SARS-COV-2 by not only the antibody-mediated immunity, but also cellular immunity.

### Example 2: Further Preparation of Exemplary Anti-Viral Nanoparticles

### To Optimize Selection of T Cell and B Cell Epitopes and Antigen Designs.

CD4 and CD8 T cell epitopes have been computationally characterized and identified CD4 and CD8 T cell epitopes from SARS-COV-2 using an ensemble of machine learning algorithms that consider conservation of viral sequences, expression level, glycosylation, structural constrains, MHC allelic distribution in the human population. The identified epitopes will be validated in vitro using peripheral blood mononuclear cells from SARS-COV-2 convalescent individuals. The validated epitopes will be constructed into mRNA for optimal expression, processing and presentation.

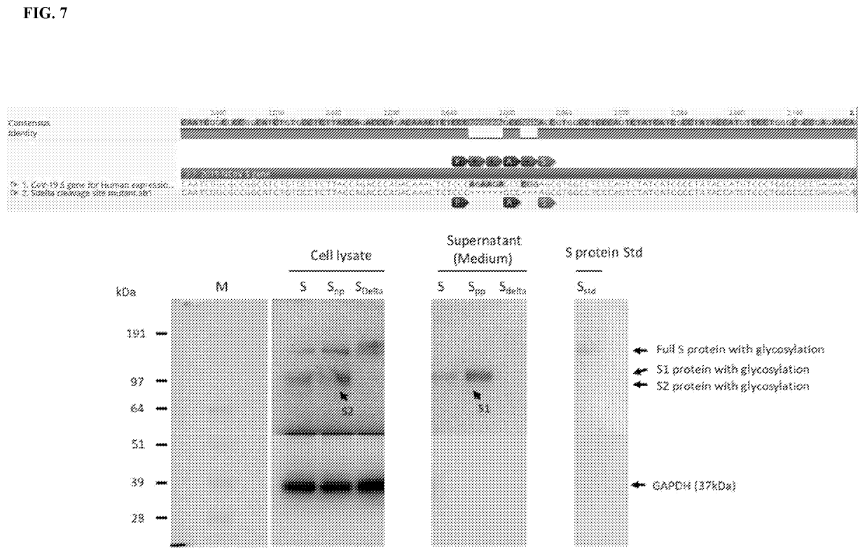

Three kinds of mRNAs candidates have been identified for vaccine design. 1. Full spike protein (S). 2. Full spike protein with two proline mutation. (Sₚₚ) 3. Full spike protein with mutated furin cleavage site. (S_{delta}). As shown in FIG. 7, Sₚₚ showed strongest expression in HEK293 cells. The cleaved S1 and S2 protein could be detected in cell lysate. Only S1 protein could be detected in supernatant. The Sₚₚ mRNA was chosen for the future experiment.

To Deliver Vaccine mRNA into Dendritic Cells in the Draining Lymph Nodes Using Novel Synthetic Lipid Nanoparticles.

A new class of imidazole containing lipids that target mRNA to DCs or draining lymph nodes have been identified. Encoding mRNA will be formulated into lipid nanoparticles (LNP) (mRNA-LNPs) using these new lipids and other known lipids and compare their delivery of mRNA into DCs in the draining lymph nodes and induction of CTL and nAb responses in HLA-A2 transgenic mice. The targeted delivery and immune responses will be optimized using DC-targeting ligands.

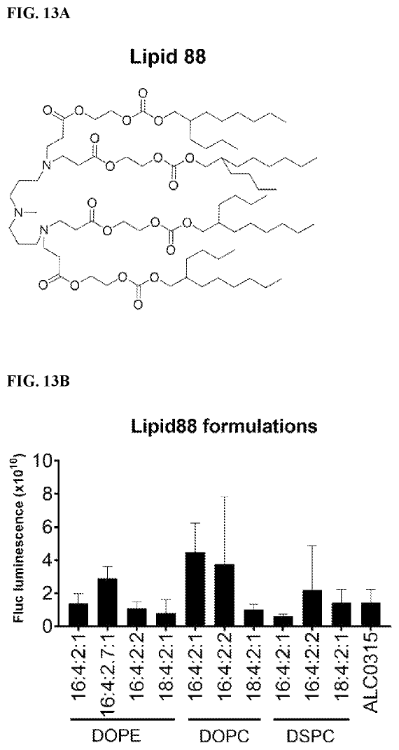

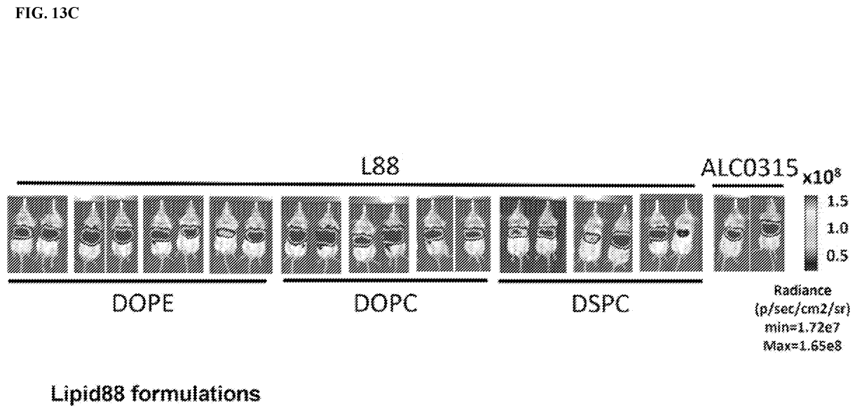

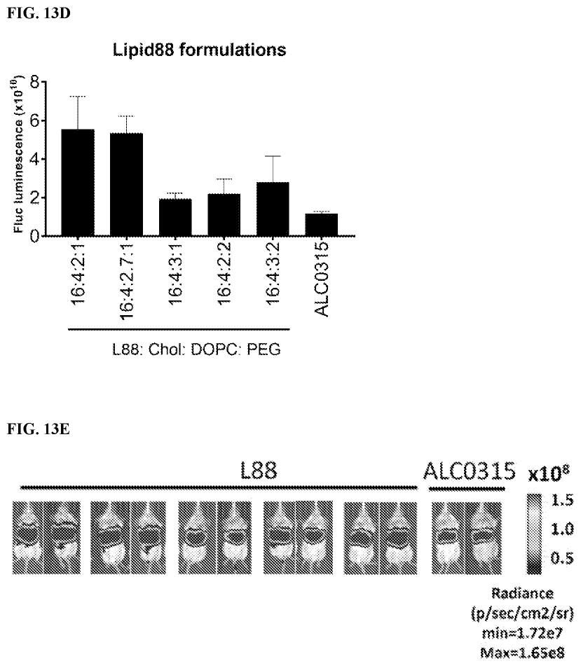

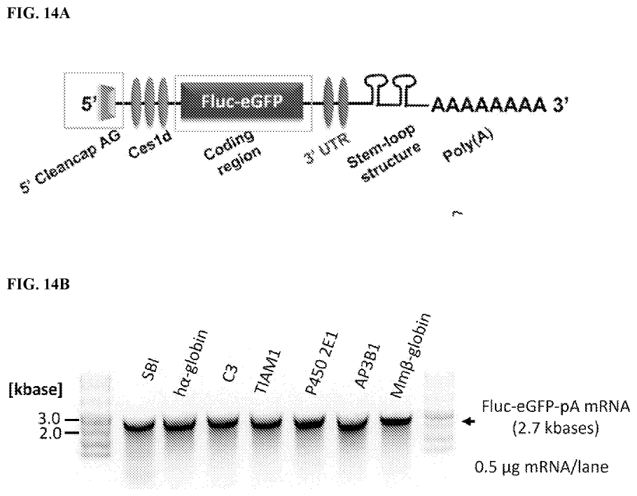

A LNP with enhanced mRNA expression in draining lymph nodes, as compared with the ALC-0315 LNP used in Pfizer-biontech's BNT162b2 mRNA vaccine, has been produced. As shown in FIG. 13, the expression of the model Luc mRNA in draining lymph nodes was stronger than the ALC-0315 formulation after 6 h of tail-base injection. Moreover, the 113 formulation showed decreased expression in liver compared with ALC-0315 formulation.

To Augment nAb Response with Toll-Like Receptor (TLR) Ligands.

TLR ligands (Pam₃CSK₄ and poly I:C) that dramatically augment nAb responses to a protein-based dengue virus vaccine while reducing non-neutralizing antibody responses have been identified. The two TLR ligands individually will be produced and in combination into mRNA-LNPs and test if they enhance nAb response and reduce antibody-dependent enhancement. The mechanisms underlying the enhanced nAb response will be investigated for further adjuvant optimization.

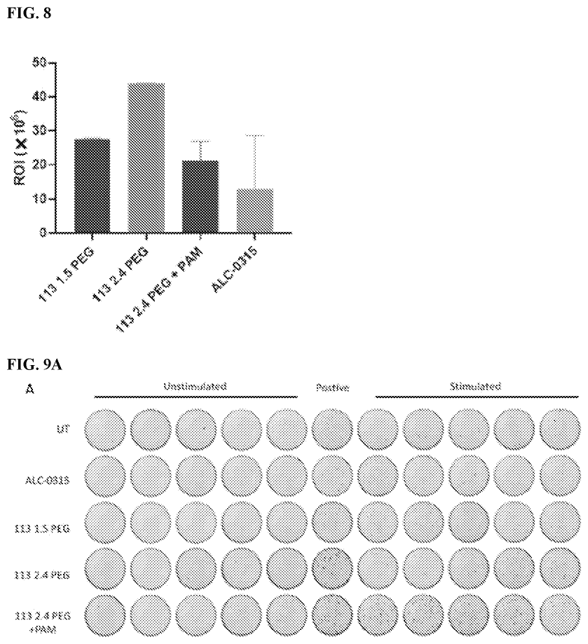

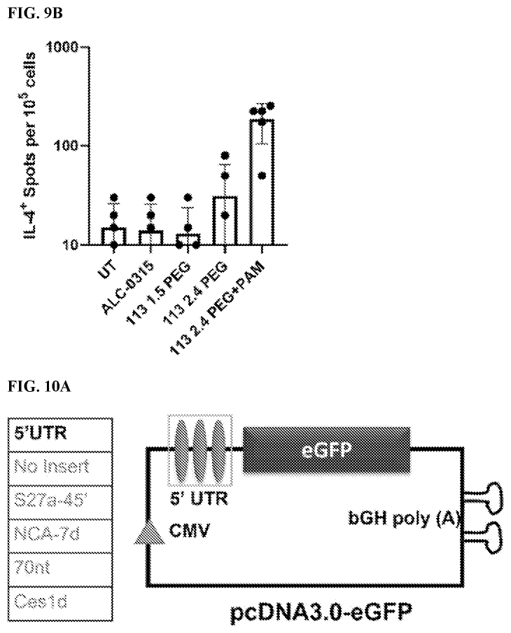

The formulations of the adjuvant contained LNPs with enhanced mRNA expression in draining lymph nodes have been optimized. Pam3CSK4 formulated at the weight ratio of 2.5% decreased the mRNA expression in draining lymph nodes, which is still similar to ALC-0315 formulation (FIG. 8).

To Determine Whether Select mRNA Vaccines Confer Protection Against SARS-COV-2 Infection in a Small Animal Model.

ACE2 transgenic mice and hamster are susceptible to SARS-COV infection. ACE2 transgenic mice or hamsters or ferrets will be vaccinated with the most optimized vaccine candidates and the induction of immune responses will be evaluated by ELISA, SARS-CoV-2 neutralization and CTL assays. Vaccinated animals will be challenged with SARS-CoV-2. Clinical signs, viral load and animal health status will be monitored. Together, these data will permit an evaluation of vaccine efficacy in the mitigation of SARS-COV-2 in a relevant pre-clinical animal model.

The antibody and T cell responses after vaccination using the 113 formulations was tested. ALC-0315 formulation was used as control. In general, our 113 formulation exhibited comparable antibody response and better T cells response compared with ALC-0315 formulation. Moreover, addition of PAM3CSK4 increased the Th1 T cell response and percentage of IL-4 secreting T cells.

The IgG, IgG1, and IgG2a antibody titers were measured by ELISA assay (FIGS. 4A-D). ALC-0315 and 113 2.4 PEG formulation showed similar antibody responses. The addition of PAM3CSK4 into the system decreased the antibody response. However, the IgG2a/IgG1 ratio was increased compared with other groups.

The T cell response after vaccination was tested by ELISpot assay. The 113 2.4 PEG formulation significantly increased the population of IFN-γ+ T cell after stimulation by S protein peptide pool. More interestingly, the addition of PAM3CSK4 significantly induced IL-4 secreting T cells compared with all other groups.

### Example 3: Preparation of Exemplary Vaccines

### Procedure and Results.

Plasmid construction pcDNA3.0-eGFP (#13031) was purchased from Addgene for Untranslated Regions (UTRs) optimization in vitro delivery. To introduce 5′UTR into the 5′ terminal of eGFP, four fragments (S27a-45′, NCA7d, 70 nt and Ces1d) were synthesized from IDT (Integrated DNA Technologies, US) and PCR amplified by primers A204-A215 (Sequences—Tables 1 and 3). Purified PCR products and pcDNA3.0-eGFP were then incubated with KpnI and EcoRI at 37ºC for 2.5 hrs to produce KpnI and EcoRI sticky ends. Each fragment was ligated with linearized pcDNA3.0-eGFP using T4 DNA ligase (NEB, US). Ligation products were then transformed into competent cells (E. coli DH5a) to produce the complete pcDNA3.0-5′UTR-eGFP construct. The 13 fragments in Sequences—Table 2 were then introduced into pcDNA3.0-Ces1d-eGFP and pcDNA3.0-70 nt-eGFP at 3′ terminal of eGFP through XbaI-ApaI sites and homolog arm fusion reactions with primers A333/A334 (Sequences—Table 3). The homolog fusion reactions were manipulated using Cold Fusion Cloning Kit (System Biosciences, US). All inserts were validated by Sanger sequencing (Genewiz, US) prior to in vitro delivery.

Modified pcDNA3.0-eGFP plasmids containing each UTRs insert was screened in HEK293T cells. Top-performing 5′UTRs and 3′UTRs were selected and introduced into pMRNA-Fluc-eGFP. Two best-performing 5′UTR fragments (Ces1d and 70 nt) were PCR amplified by primers A222-A225 and then ligated to KpnI-EcoRI sticky ends at 5′ terminal of Fluc-eGFP using T4 DNA ligase (Sequences—Table 3). Screened 3′UTR fragments from pcDNA-Ces1d-Fluc-eGFP-3′UTR, Complement Component (C3), WIPI2, human a-globin, TIAM1, Cytochrome P450 2E1 (P450 2E1) and AP3B1, were incorporated into the 3′ terminal of Fluc-eGFP at the SspI site of pMRNA-Fluc-eGFP using primers A348-A351 (Sequences—Table 2 and 3). Similarly, based on pcDNA-70 nt-Fluc-eGFP-3′UTR in vitro optimization, eight fragments (S0_M_T1012, human a-globin, Apolipoprotein A-II, OXR1, POTEE, MS10433, AP3B1 and YY2 Transcription Factor) were also chosen as potential candidates and introduced into pMRNA-70 nt-Fluc-eGFP at the SspI site using primers A348-A351 (Sequences—Table 2 and 3). Resulted pMRNA-Ces1d-Fluc-eGFP-3′UTR and pMRNA-70 nt-Fluc-eGFP-3′UTR products were used as templates for tail PCR reaction

Tail PCR The pMRNA-Ces1d-Fluc-eGFP-3′UTR and pMRNA-70 nt-Fluc-eGFP-3′UTR plasmids were used as templates for gene polyadenylation using the Tail PCR Primer A93/A95, where the reverse primer contains 120 oligo(dT). (Sequences—Table 3) The Tail PCR reaction (50 μL) was performed in a NEBR High-Fidelity PCR master mixture (New England Biolab Inc, US): 2× High Fidelity Buffer (1×), Tail PCR primer A93/A95 (0.5 μM), plasmid template (2 ng/μL). The reaction was then applied to a PCR program: 98° C. 30 s, 98° C. 10 s, 64° C. 30 s, 72° C. 1.5 min, 72° C. 10 min, 4° C. hold for 40 cycles. The PCR mixture was then treated with Proteinase K (200 μg/mL) for one hour to digest RNases. The RNase-free Tail PCR products were purified with a Monarch Gel Purification Kit (New England Biolab Inc, US). The purified products were measured on a Nanodrop and used as a template for in vitro transcription.

In Vitro Transcription of mRNA

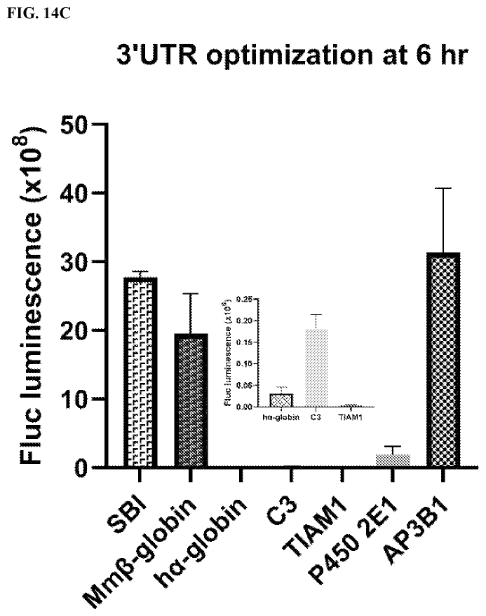

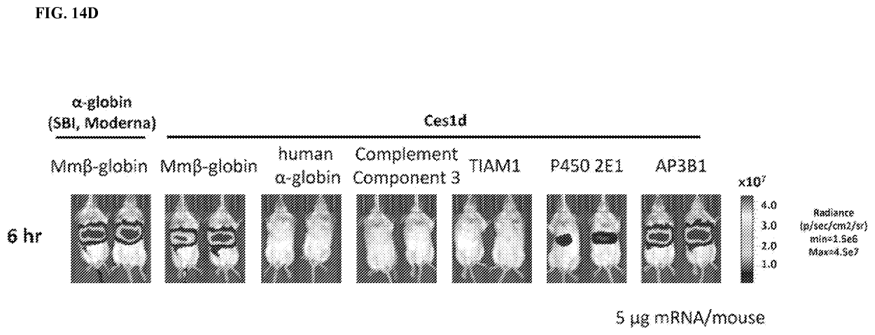

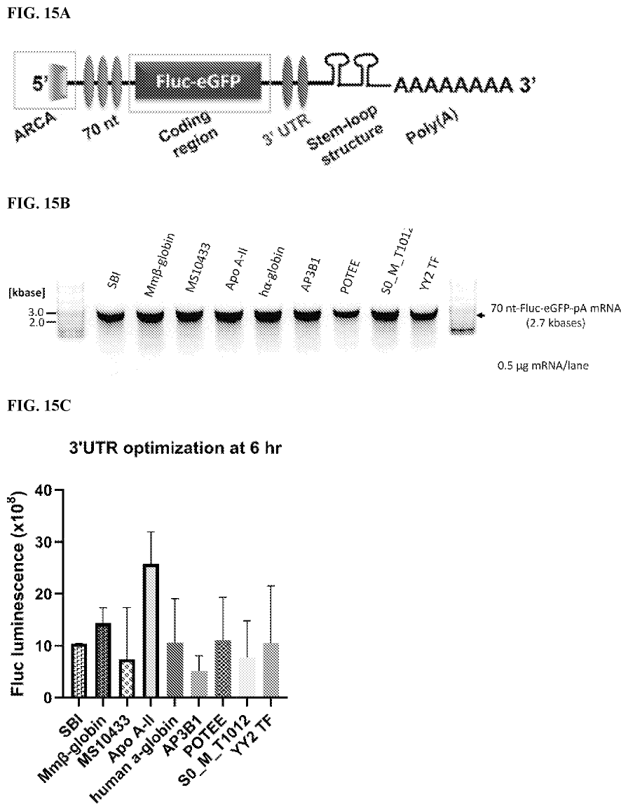

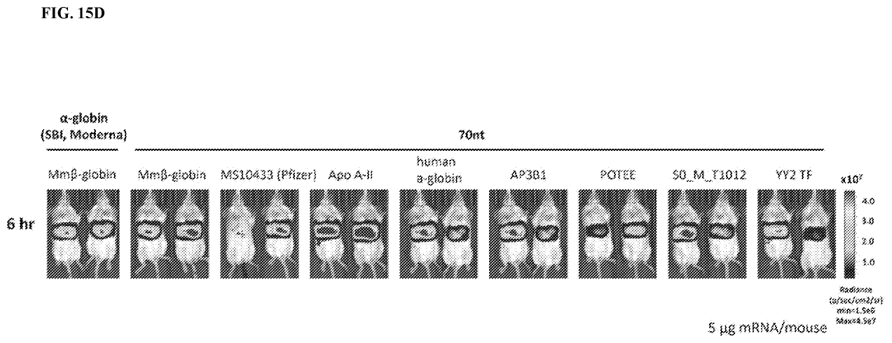

Tail PCR products were used as the template for IVT-mRNA synthesis. Modified Ces1d-Fluc-eGFP-3′UTR and 70 nt-Fluc-eGFP-3′UTR mRNA was generated by T7 polymerase through an in vitro transcription reaction [10×T7 reaction buffer (1×), ARCA cap (10 mM), ATP (10 mM), CTP (10 mM), GTP (3.75 mM), N¹mψ (3.75 mM), template (0.5 μg) and T7 RNA polymerase mix], treated with DNase I and Antarctic Phosphatase (New England Biolabs, US) and purified using a MegaClear Kit (Life Technologies, US). The purified product concentrations were measured on a Nanodrop and stored at −80° C. All mRNA quality was further validated on 1% agarose gels (RNase-free). Results are shown in FIGS. 14B and 15B.

### Lipid 88 Formulations Optimization in Mice Liver.

All animal procedures were approved by the Tufts University Institutional Animal Care and Use Committee (IACUC) and performed with ethical compliance at Tufts University. 6-week-old female BALB/c mice (Charles River, US) were used for the in vivo screening of Lipid 88 formulations. Lipid 88 (L88) was synthesized through Michael addition reactions, which add an amine head of 306 to multiple hydrophobic carbon tails termed as tail 88 (FIG. 13A). To screen these formulations for high delivery efficacy, TriLink 5moU-Fluc mRNA was used as model cargo. Briefly, lipid 88, cholesterol (Chol), phospholipids (DOPE, DOPC and DSPC,), and DMG-PEG2000 (Avanti Polar Lipids, US) were dissolved in 100% ethanol at gradient weight ratios of 16/4/2/1 to 18/4/2/1 at a final lipidoid concentration of 10 mg/mL. The mixture of active lipids, helper lipids, and ethanol was added to a triple volume of 25 mM sodium acetate buffer (pH 5.2) dropwise, stirring by vortex. Formulated LNPs were dialyzed with a Thermo Scientific™ Slide-A-Lyzer MINI dialysis device (3.5K MWCO) overnight. The Lipid 88/Fluc mRNA was prepared by simply mixing each blank lipid 88 formulation and Fluc mRNA at a weight ratio of 10:1 in PBS solution, pH7.2. 5moU-FLuc mRNA/LNPs was intravenously injected into the mice at a dose of 0.2 mg/kg. 6 hrs post injection, mice were intraperitoneally injected with 100 μL of D-Luciferin potassium salt (GoldBio) solution (15 mg/mL in PBS), anesthetized under isoflurane anesthesia, and levels of Fluc expression were evaluated using IVIS imaging system (Caliper Life Sciences, US). As shown in FIGS. 13B-13E, mRNA delivery with multiple formulations of lipid 88/helper lipids resulted in more intense luciferase bioluminescence emission as compared to the commercial lipid ALC0315 (Pfizer). In vivo images of living mice showed that Lipid 88 targeted Fluc mRNA delivery to the liver, with a weight ratio of 16:4:2:1 (L88:Chol:DOPC:DMG-PEG) as the top-performing formulation with the highest delivery efficiency in mice liver (FIG. 13).

mRNA Transfection Experiments

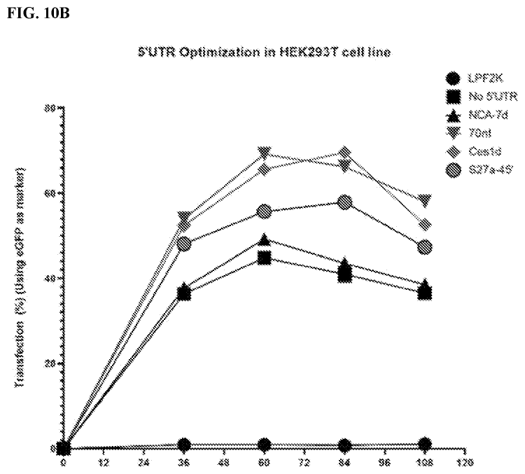

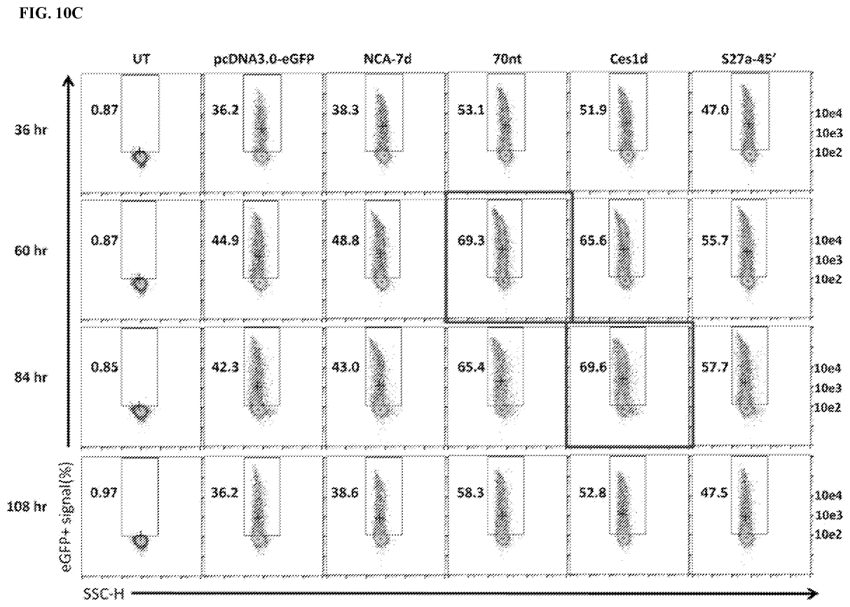

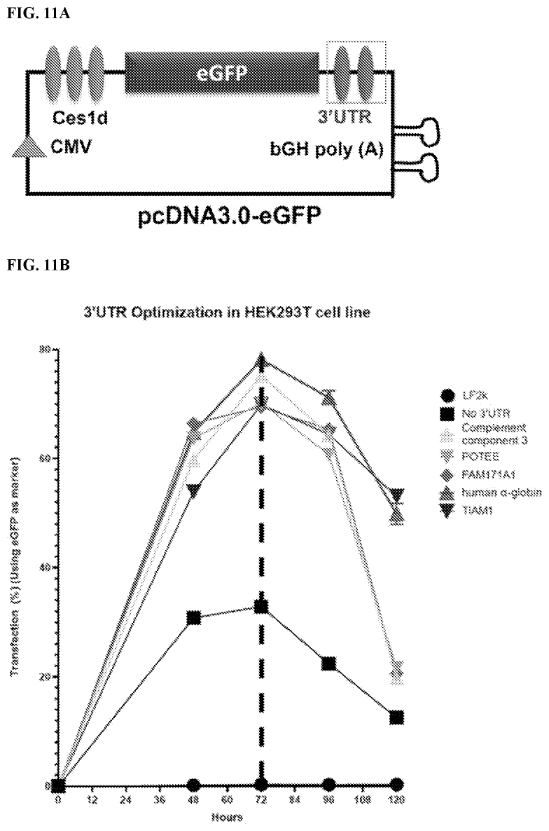

5′UTR optimization in HEK293T cells. Prior to transfection, HEK293T cells were seeded on a 48-well plate at 40,000 cells/well, with 500 μL DMEM culture in each well. 6 groups were set with different treatments: Lipofectamine 2000 (LPF2k) only, pcDNA3.0-eGFP, pcDNA3.0-NCA7d-eGFP, pcDNA-70nt-eGFP, pcDNA3.0-Ces1d-eGFP and pcDNA3.0-S27a45′-eGFP. LPF2k was complexed each plasmid in equal amount (0.5 μg/0.5 μg) before being added to each well. At each time point (36 hrs, 60 hrs, 84 hrs and 108 hrs), cells were washed with PBS and then treated with 500 μL 0.25% Typsine-EDTA 10 (Gibco) for 3 min. Resuspended cells were applied to the Attune NxT Flow Cytometer (Thermo Fisher) directly (FIG. 10). Ces1d and 70nt were selected as the top-performing 5′UTR candidates using pcDNA3.0-eGFP transfection.

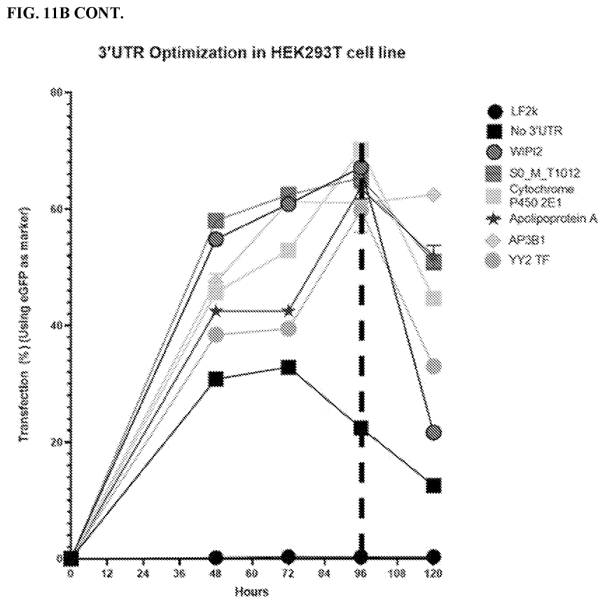

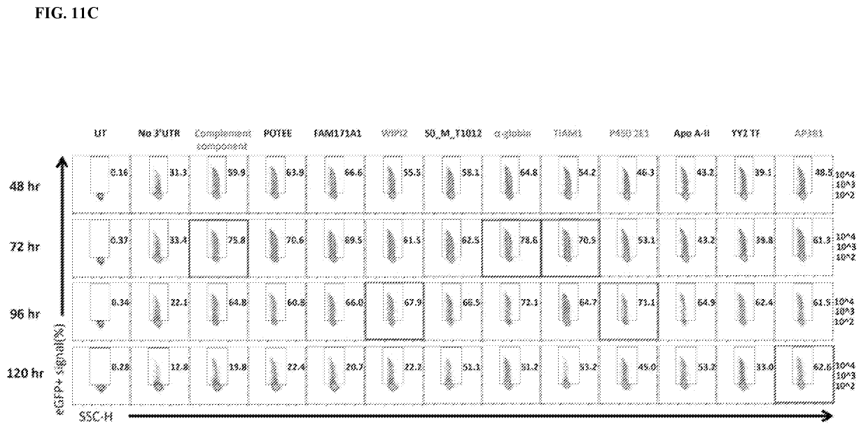

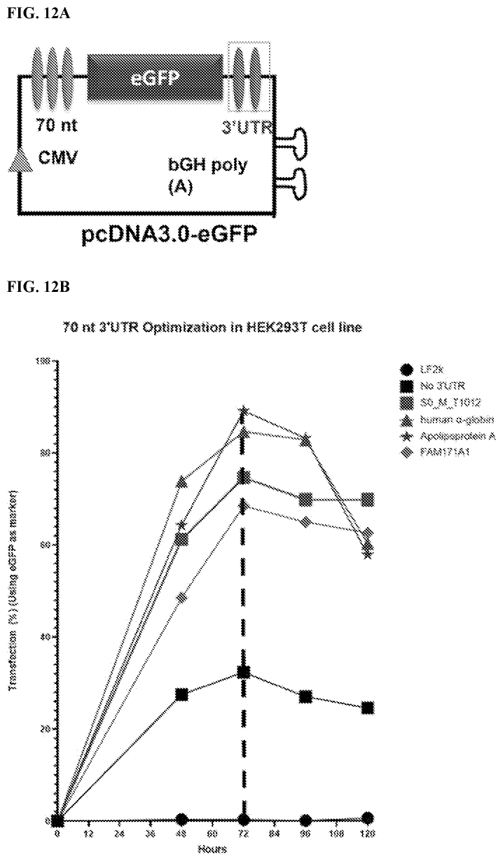

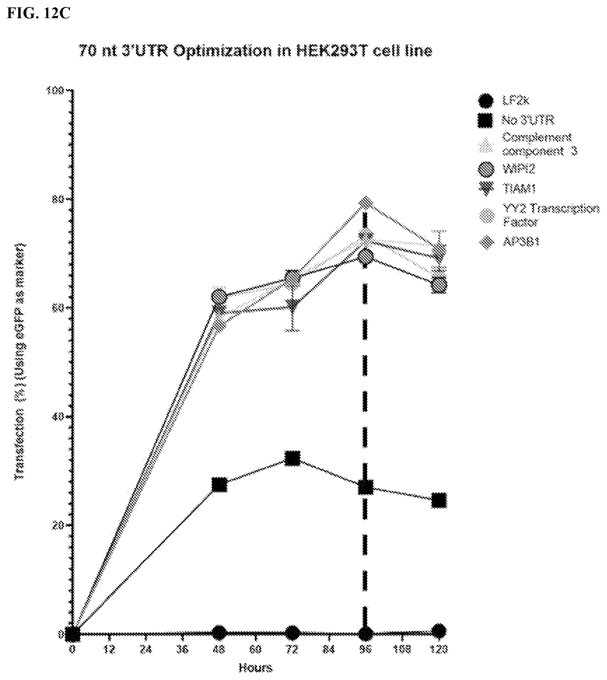

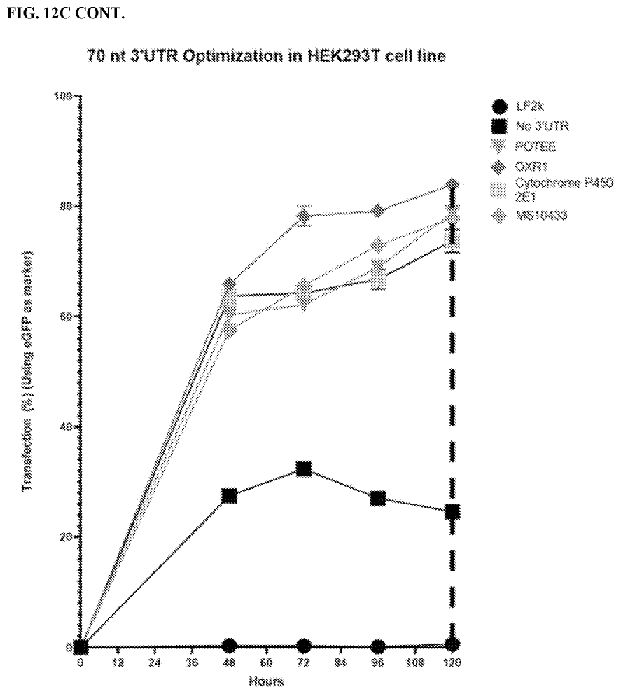

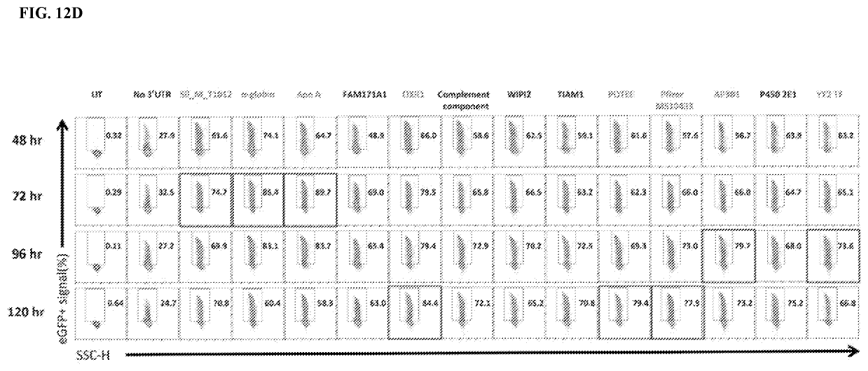

3′UTR optimization in HEK293T cell. Similar to 5′UTR optimization, thirteen 3′UTRs modified pcDNA3.0-Ces1d-eGFP and pcDNA3.0-70nt-eGFP were encapsulated in LPF2k at a 1:1 weight ratio and then transfected into HEK293T cells. At each time point (48 hrs, 72 hrs, 96 hrs and 120 hrs), cells were washed with PBS and then treated with 500 μL 0.25% Typsine-EDTA (Gibco) for 3 min. Resuspended cell were applied to the Attune NxT Flow Cytometer (Thermo Fisher) directly (FIG. 11). In pcDNA3.0-Ces1d-eGFP delivery, Complement Component (C3), WIPI2, human a-globin, TIAM1, Cytochrome P450 2E1 (P450 2E1) and AP3B1 were the best-performing 3′UTR insert and resulted in higher eGFP expression. On the other hand, S0_M_T1012, human a-globin, Apolipoprotein A-II, OXR1, POTEE, MS10433, AP3B1 and YY2 Transcription Factor were the top-performing 3′UTR candidates for pcDNA3.0-70nt-eGFP expression (FIGS. 2 and 3).

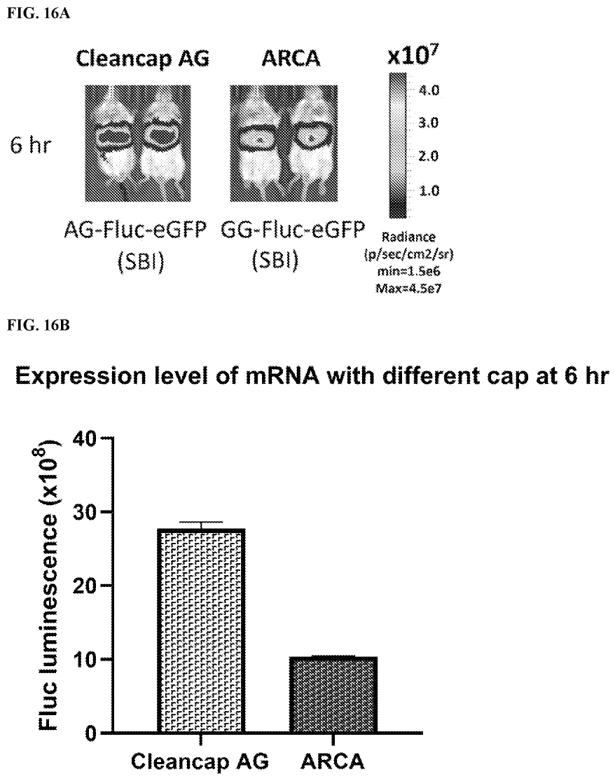

Mouse liver-directed 3′UTR screening in mRNA format. 6-week-old female BALB/c mice were used for in vivo modified Fluc-eGFP mRNA screening with L88 LNP delivery. Each UTRs modified Fluc-eGFP mRNA were encapsulated in an optimal Lipid 88 formulation (L88:Chol:DOPC:DMG-PEG=16:4:2:1, w w) at the weight ratio of 1:10 in PBS solution (RNase-free). The L88/Fluc-eGFP mRNA complex was intravenously injected into the mice at a mRNA dose of 0.2 mg/kg. At each preset time point, mice were injected with 100 μL of D-Luciferin potassium salt (GoldBio) solution (15 mg/mL in PBS), anesthetized under isoflurane anesthesia, and evaluated using IVIS imaging system (Caliper Life Sciences, US). The IVIS results clarified that fragment AP3B1 (S115 in Sequences—Table 2) outperformed other six 3′UTR candidates in CleanCap AG-Ces1d-Fluc-cGFP mRNA expression in mice livers under Lipid 88 delivery (FIG. 14). In ARCA capped 70nt-Fluc-eGFP mRNA expression, Apolipoprotein A-II (Apo A-II) (S109 in Sequences—Table 2) substitution produced the highest Fluc expression in the liver relative to other eight 3′UTR modifications (FIG. 15). Using the same mRNA construct, we found that CleanCap AG capped Fluc-cGFP mRNA resulted in approximately 3-fold higher luminescence intensity compared to the ARCA capped mRNA (FIG. 16).

### Example 4: Preparation of Exemplary Anti-Cancer Nanoparticles

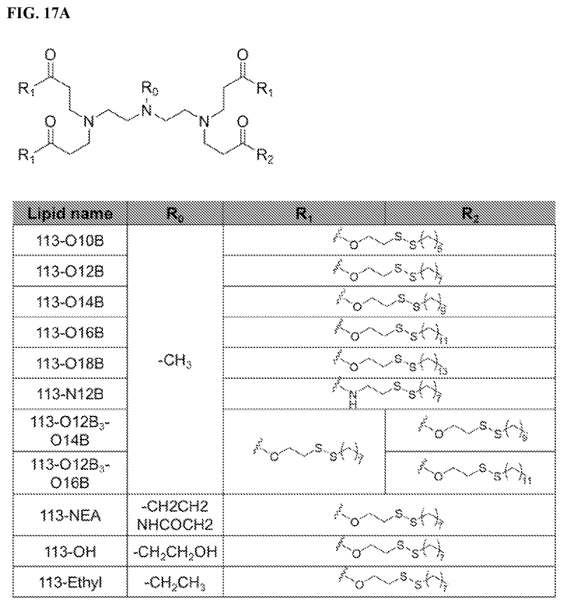

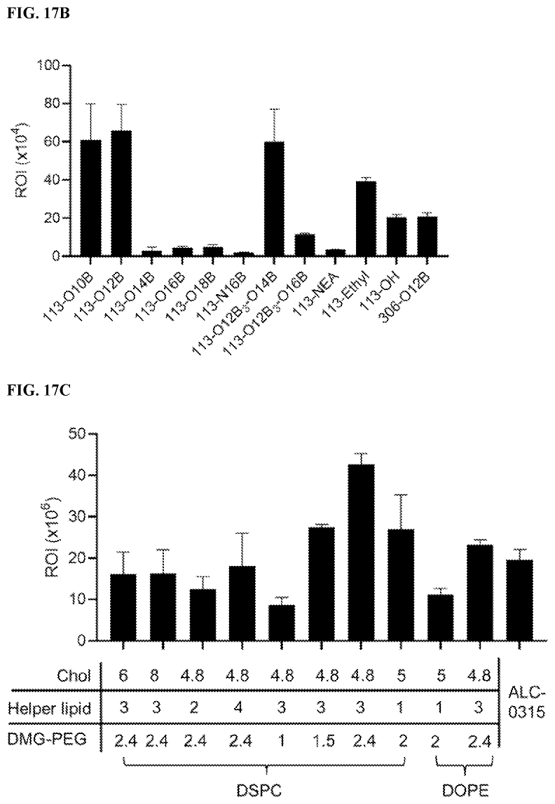

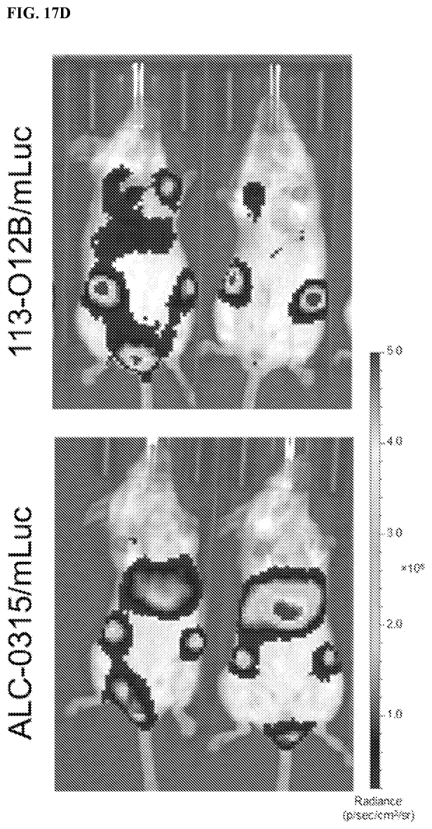

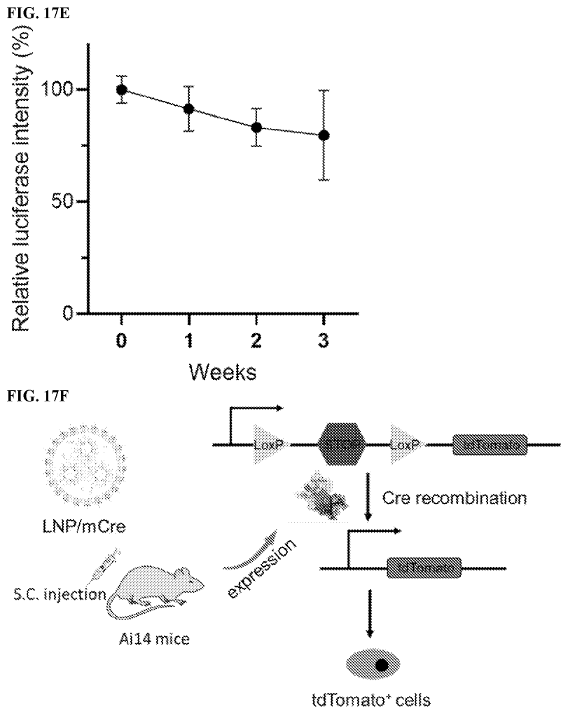

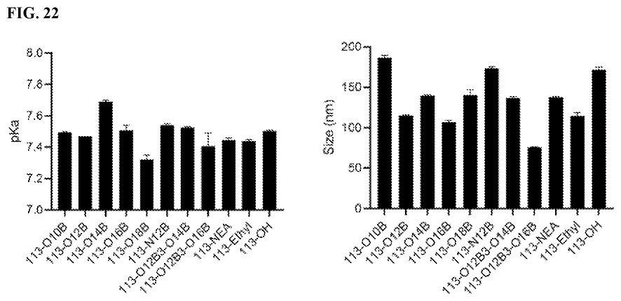

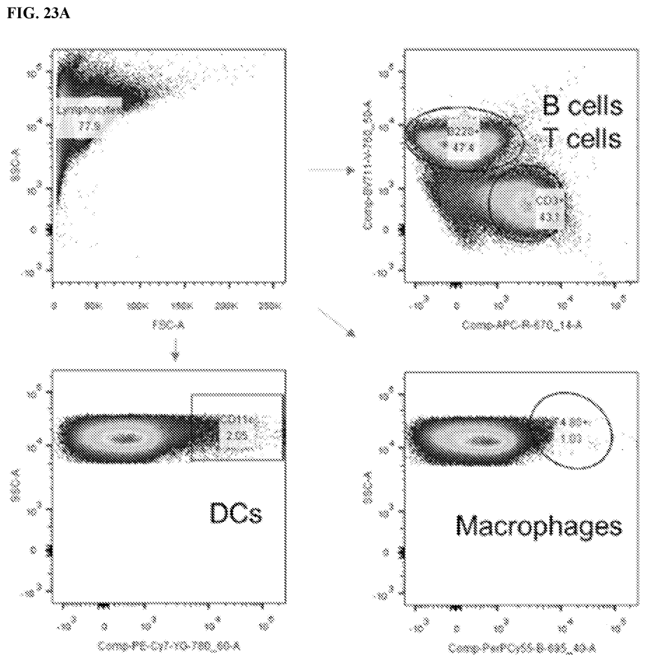

The LNPs used for screening were synthesis via the Michael addition of amine-bearing head and acryloyl group containing aliphatic chain. A library of reduction responsive lipids was developed. The library is based on structures of the head, tail, and linker, such as side group in head amine, linker type, tail length, and tail combination (FIG. 17A). The LNPs were formulated with cholesterol (Chol), dioleoylphosphatidylcholine (DOPC), and 1,2-dimyristoyl-rac-glycero-3-methoxypolyethylene glycol-2000 (DMG-PEG) in the weight ratio of 16:4:2:1. Luciferase mRNA (mLuc) was used as a model mRNA for tracking the in vivo distribution of the expressed protein. The total intensity within LN after s.c. injection of LNP/Luc mRNA for 6 h was calculated in FIG. 17B. First, the length of the tail made great difference in the expression of mRNA in LN. The shorter length of 010B and O12B exhibited better expression, while the longer length with 2 more carbons showed almost no efficacy at all. The combination of tails with different length also proved the importance of tail length, that is, the combination of longer tail in the lipid decreased the delivery efficacy. Second, the easter linker played an important role in the delivery. The replacement of easter bond to amide bond showed no transfection effect in LNs. Third, changing the methyl groups of amine head to alcohol, ethyl, and N-(1,2-ethanediyl)acetamide (NEA) group reduced the signal to some extent. 113-O12B was selected as the top lipid for mRNA delivery to LNs in this library. Further experiments including the pKa and size of LNPs were analyzed to explore the in-depth relationship between the properties of LNPs and mRNA expression (FIG. 22). However, no obvious conclusion could be drawn, which might be resulted from the limited numbers of samples.

The formulation of 113-O12B was further optimized to get best delivery efficacy. ALC-0315 that applied in Pfizer/biotech's Covid-19 mRNA vaccine was selected for comparison. As shown in FIG. 17C, the component of Chol, DOPC, and DMG-PEG all had impacts on the mRNA transfection in LNs. The optimized weight ratio of the four components was determined to be 16:4.8:3:2.4, showing better signal in LNs compared with ALC-0315/mLuc. The detailed pictures of the luminescence distribution in mice after s.c. injection of LNP/Luc mRNA were present in FIG. 17D. The obvious signal in draining LNs could be observed in both 113-O12B and ALC-0315 LNPs, while ALC-0315 LNP showed significantly higher expression in liver, confirming the LN-targeted delivery of 113-O12B. Moreover, the stability of LNP/mRNA formulations at 4° C. was evaluated in FIG. 17E. The 113-O12B/mLuc maintained 75% signal after stored at 4° C. for three weeks compared with the fresh prepared one, demonstrating the acceptable stability and flexible storage condition of 113-O12B.

To explore the detailed cell type that could be transfected by LNP within LNs, the gene engineered Ai14 mice were applied owing to the activatable and stable signal expression after mRNA expression (FIG. 17F). In detail, the gene encoding the red florescence protein tdTomato was blocked by stop gene between two LoxP segments. When the mRNA encoding Cre mRNA was delivered and expressed within cells, the LoxP gene was cut into DNA cycle. Afterwards, the tdTomato gene was activated and the protein was expressed with red fluorescence signal, which could be detected by flow cytometry. As shown in FIG. 17G, both 113-O12B and ALC-0315 successfully delivered Cre mRNA to antigen-presenting cells (APC) including macrophages and dendritic cells (DCs). 113-O12B showed better mRNA expression of ˜25% in DCs and ˜35% in macrophages, which was important to the following activation of adaptive immunity. Decreasing the amount of DMG-PEG lowered the mRNA expression in all cells, suggesting the important function of PEG for in vivo delivery.

### 113-O12B/OVA Elicited Robust CD8+ T Cell Response and Prevention Against B16F10-OVA Tumor Model

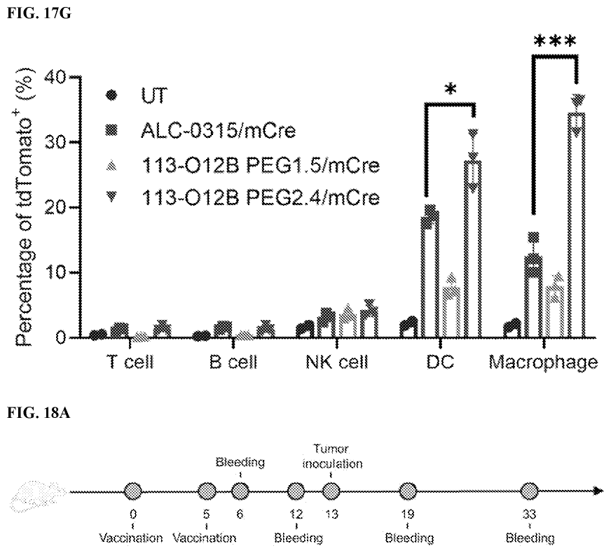

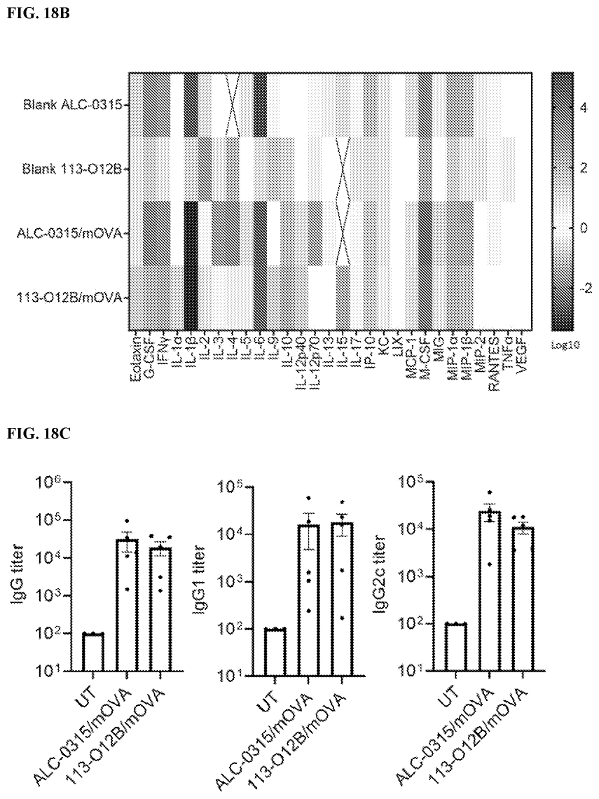

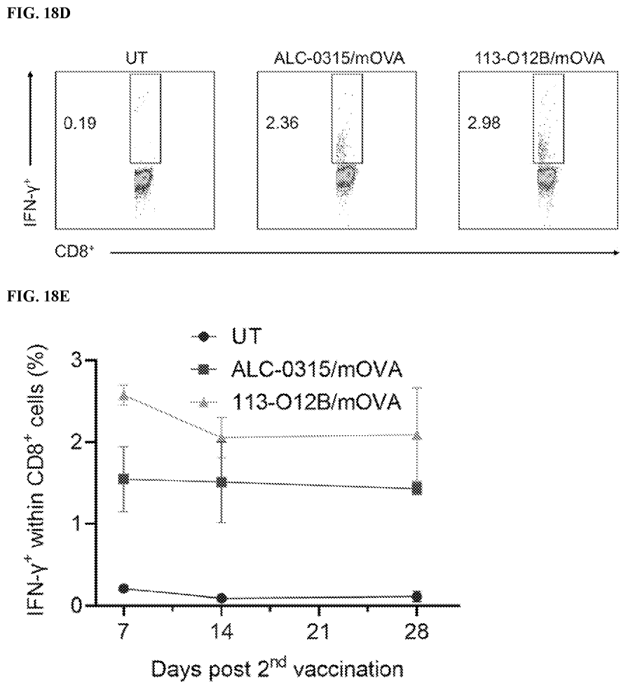

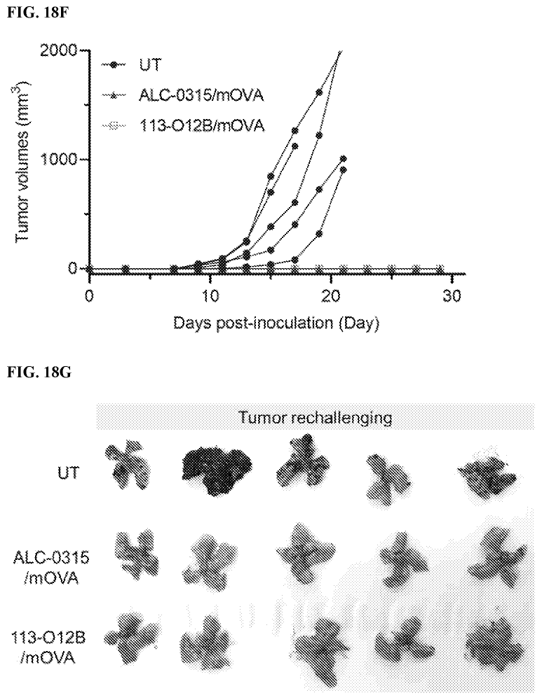

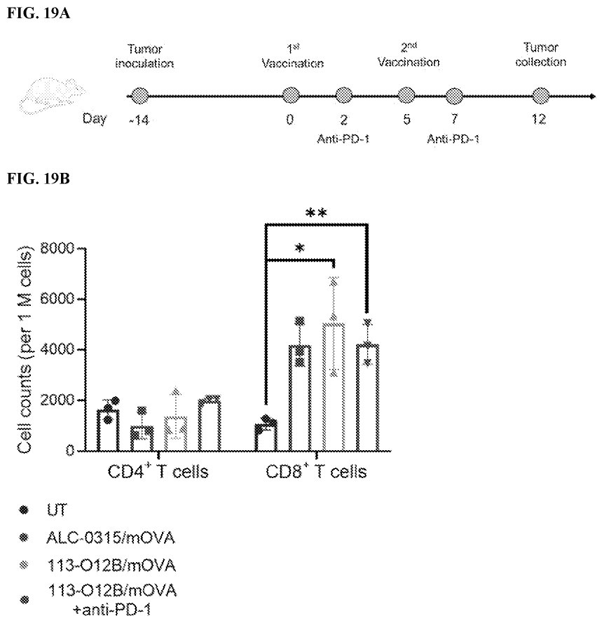

OVA was chosen as a model antigen and OVA-transduced B16F10 (B16F10-OVA) cells were used as the model tumor cells. The vaccination followed the timeline in FIG. 18A with the prime dose on day 0 and the boost dose on day 5. The levels of cytokines and chemokines after the treatment of blank LNPs and LNP/mRNA for 24 h were shown in FIG. 18B. Blank ALC-0315 showed stronger activation of innate immune system with the significantly increased cytokine levels such as G-CSF, M-CSF, IFN-γ, and IL-6 and chemokine levels including MCP-1, MIP-1α, and MIP-1β. The formulation with OVA mRNA increased the cute inflammatory response owing to the expression of the foreign protein OVA. 113-O12B/mOVA showed comparable cytokine and chemokine levels compared with those of ALC-0315/mOVA, suggesting the successful activation of innate immune system by rapid expression of OVA. More important, IL-6, which played important role in the proliferation and differentiation of T cells for adaptive immunity, was improved significantly in 113-O12B/mOVA treated groups compared with the blank 113-O12B, probably resulting from the targeted delivery of mRNA to LNs.

The antibody levels of both mRNA vaccines were evaluated after 14 days of second dose (FIG. 18C). Three different types of IgG including total IgG, IgG 1, and IgG2c were measured by enzyme-linked immunosorbent assay (ELSIA). 113-O12B/mOVA showed comparable antibody response of all three types of antibodies compared with those induced by ALC-0315/mOVA. However, the peak antibody level was observed in ALC-0315 group, which might be resulted from the high expression of OVA protein in liver. The percentage of OVA-specific CD8⁺ T cells was evaluated by intracellular cytokine staining. As shown in FIGS. 18D & 18E, after 14 days of the second vaccination, the percentage of IFN-γ⁺ cells within CD8⁺ T cells stimulated by OVA peptide (SIINFEKL) in 113-O12B group reached to about 2.57%, which was significantly higher than that of ALC-0315 group (˜1.55%). Moreover, the percentage of IFN-γ+ cells remain above 2% in 113-O12B group after four weeks post the second vaccination (FIG. 18E), showing the long-lasting T cells response induced by mRNA vaccine.

The protection effect of the mRNA vaccine was evaluated in B16F10-OVA xenograft model. 1 million tumor cells were injected subcutaneously at the right flank of the control or vaccinated mice on day 13. As shown in FIG. 18F, the tumor grew rapidly in the mice without vaccination, which reached to endpoint within 20 days. However, no obvious tumor could be observed in both the 113-O12B/OVA and ALC-0315/OVA vaccinated mice, indicating the superior protection effect of the mRNA vaccines. To further confirm this, the metastatic model was established by the intravenous injection of 1 million cells into the control and vaccinated mice. After 20 days of the injection, the mice were sacrificed, and the lung was isolated for analysis. As shown in FIG. 18G, 4/5 mice without treatment showed obvious metastatic nodules, while none of mice in both vaccinated mice had apparent metastatic nodules. All these results demonstrated that the LNP/OVA had excellent protection effect to B16F10-OVA tumor model.

### 113-O12B/OVA Shift the Immunocellular Composition in Established B16F10-OVA Tumor Model

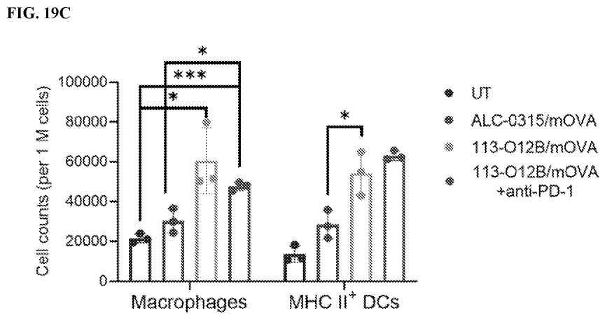

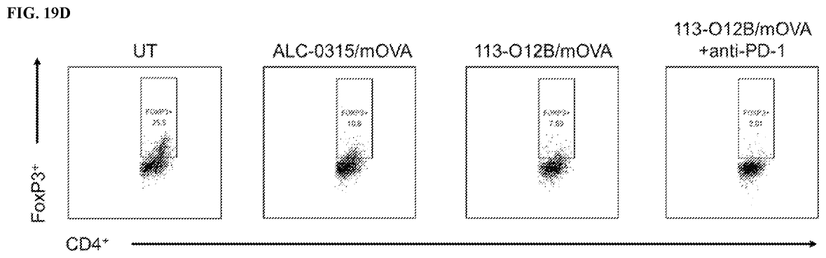

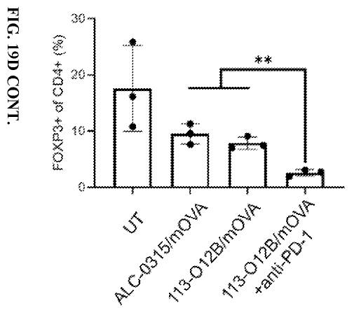

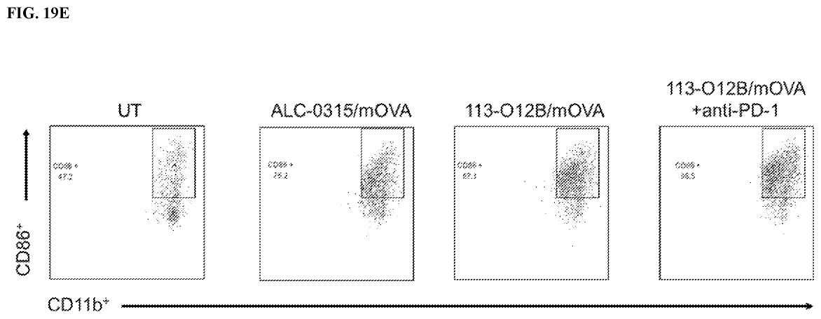

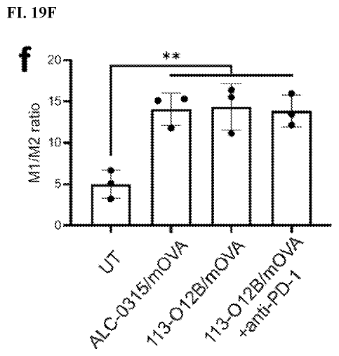

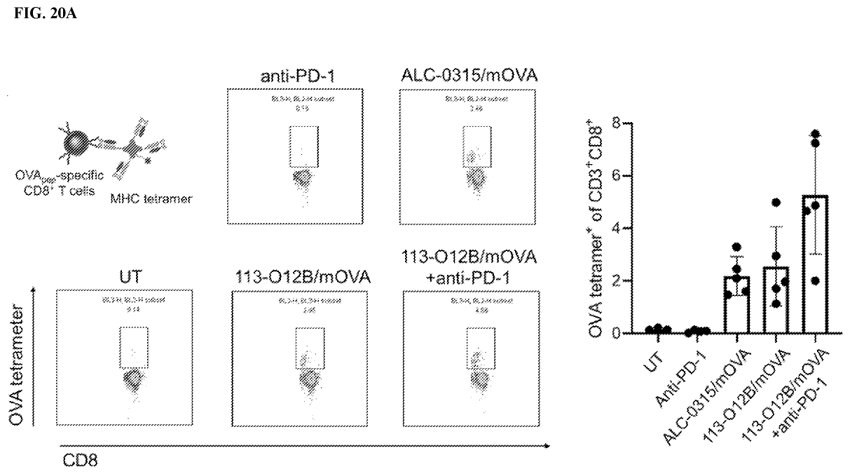

The impact of mRNA vaccine on immunocellular composition of established tumor was studied in B16F10-OVA xenograft model. 1 million B16F10-OVA cells were inoculated subcutaneously on day −14. Two weeks later, the mice received the prime and the vaccinations on day 0 and day 5, respectively (FIG. 19A). To inhibit the immune escape, the check point inhibitor anti-PD-1 antibody was applied on day 2 and 7. Tumors were collected on day 12, that is one week after the second dose, and analyzed by flow cytometry in FIGS. 19B and 19C. All the mice vaccinated with mRNA exhibited increased number of CD8⁺ T cells within tumor compared with untreated group, while no difference in CD4⁺ T cells (FIG. 19B). Interestingly, 113-O12B/OVA group showed better infiltration of macrophages and activated DCs within tumor than ALC-0315 group, suggesting a better therapeutic effect (FIG. 19C). The promoted migration of both CD8⁺ T cells and APCs was important to the therapeutic outcome of mRNA vaccine. Moreover, the combination of anti-PD-1 didn't get obvious difference in the cell numbers of T cells and APCs compared with those in 113-O12B group.

To further understand the subcellular types of the infiltrated T cells, the CD4+ T cells were stained with FoxP3, which distinguished regulatory T (Treg) cells and conventional T helper (Th) cells (FIG. 19D). About 18% of CD4+ cells were determined to be Treg cells in the tumors of untreated mice, indicating a strong immunosuppressive environment of B16F10-OVA tumor (FIG. 19D). After the two doses of vaccination, both of Treg cells in 113-O12B/OVA and ALC-0315/OVA groups decreased to about 10%, contributing to the activation of innate and adaptive response. Fortunately, the treatment of anti-PD-1 significantly reduced the percentage of Treg cells compared with 113-O12B/OVA group. Similar phenomenon was observed in many other works, indicating the combination of anti-PD1 was critical for overcoming tumor immunosuppressive.

The polarization of macrophages is also important to anti-tumor immunity. In detail, M1 macrophage benefits anti-tumor response while M2 macrophage shows suppression of adaptive immunity. M1 macrophages were marked as F4/80⁺, CD11b⁺, and CD86⁺ and M2 macrophages were labeled with F4/80⁺, CD11b⁺, and CD163⁺. As shown in FIG. 19E, less than 50% of macrophages were M1 macrophages in untreated groups. However, after the 2 doses of vaccination, the percentage of M1 type increased to more than 80%. However, the percentages of M2 macrophages were similar in all groups, remaining under 20%. The ratios of M1/M2 macrophages increased significantly in all vaccinated mice compared with that of untreated group. The driven polarization to M1 macrophages by vaccination further indicated the generation of strong anti-tumor immunity.

### Therapeutic Effect of 113-O12B/OVA to the Established B16F10-OVA Xenograft Tumor

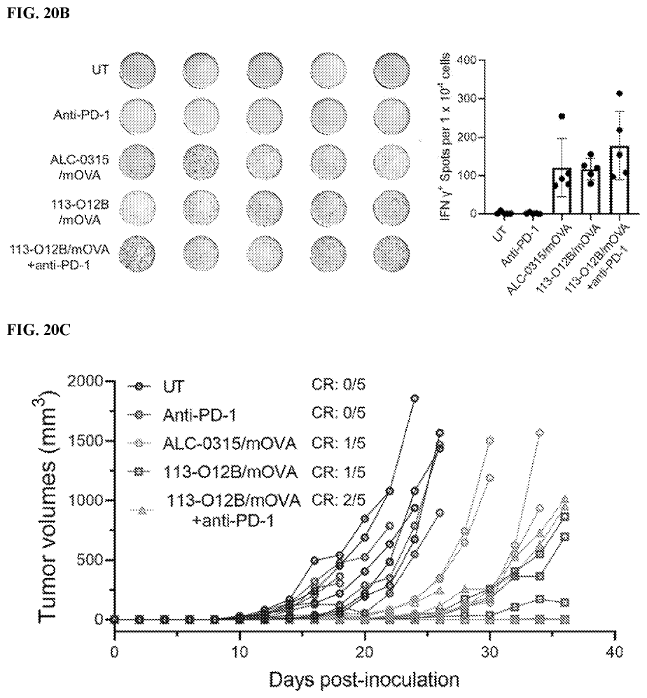

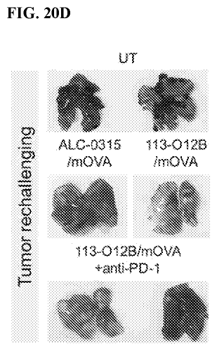

The therapeutic effect of 113-O12B-based mRNA vaccine was firstly evaluated in B16F10-OVA model. 1 million B16F10-OVA cells were injected subcutaneously at the right flank of C57/BL6 mice. After the tumor inoculation, three groups of mice received the prime and boost dose of mRNA vaccine on day 5 and 12. Anti-PD-1 antibody was administrated by intraperitoneal injection with or without 113-O12B/OVA on day 7, 11, and 15. First, the percentage of SIINFEKL-specific CD8⁺ T cells was measured on day 19 using the isolated PBMC. In FIG. 20A, almost no SIINFEKL-specific CD8⁺ T cells could be detected, while the vaccination with ALC-0315/OVA and 113-O12B/OVA increased the percentage to 2.2% and 2.5%, respectively, revealing the generation of tumor-killing CD8+ T cells after vaccination. Moreover, the combination with anti-PD-1 further upregulated the percentage to 5.3%, suggesting the important role of check point inhibitor. The levels of IFN-γ secreting T cells were evaluated by ELISpot assay in FIG. 20B. Similar to the percentage of SIINFEKL-specific CD8″ T, the mice without vaccination showed no response to the stimulation of SIINFEKL. However, all the vaccinated groups generated IFN-γ secreting T cells to some extent, providing the robust T cells response generated by mRNA vaccine.

The tumor volumes after different treatment were shown in FIG. 20C. The untreated mice exhibited a rapid growth of tumor, reaching to endpoint within 25 days (CR=0/5). Administration of anti-PD-1 only didn't relief the tumor burden at all (CR=0/5), resulting from the rapid growth of melanoma. However, the treatment of ALC-0315/OVA improved the inhibition effect with the eradication of tumor in one mouse, while rapid growth of tumor still happened in 2 of 5 mice. 113-O12B/OVA exhibited better tumor inhibition compared with that of ALC-0315. All the mice survived longer than 35 days and one of the mice showed no tumor during the whole treatment. The combination of anti-PD-1 didn't significantly improve the overall tumor inhibition but increased the CR to 2/5, which might be caused by the individual difference in response to the anti-PD-1 therapy. To further evaluate the long-term immune memory of the mRNA vaccine, the mice without tumor for 30 days were treated with 1 million B16F10-OVA cells by i.v. injection. After 18 days of the injection, the lungs of the mice were collected and pictured in FIG. 20D. Obvious metastatic nodules could be found in the lung of the mice without vaccination. However, all the mice survived in the therapeutic experiment showed no metastatic tumor at all, demonstrating the generation of long-term immune response.

### Therapeutic Effect of 113-O12B/TRP2₁₈₀₋₁₈₈ to the Established B16F10 Xenograft Tumor

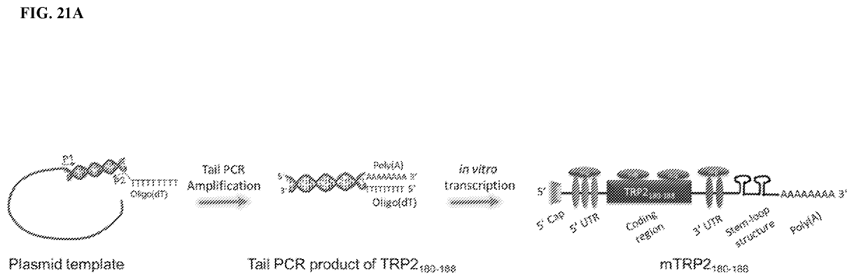

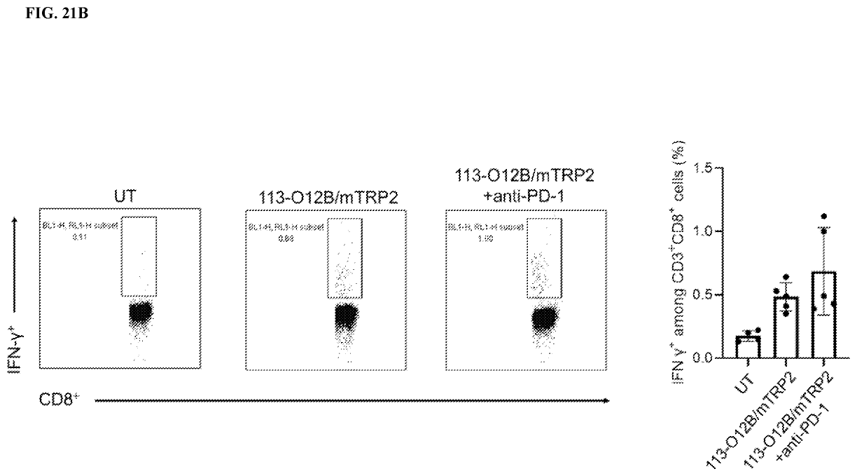

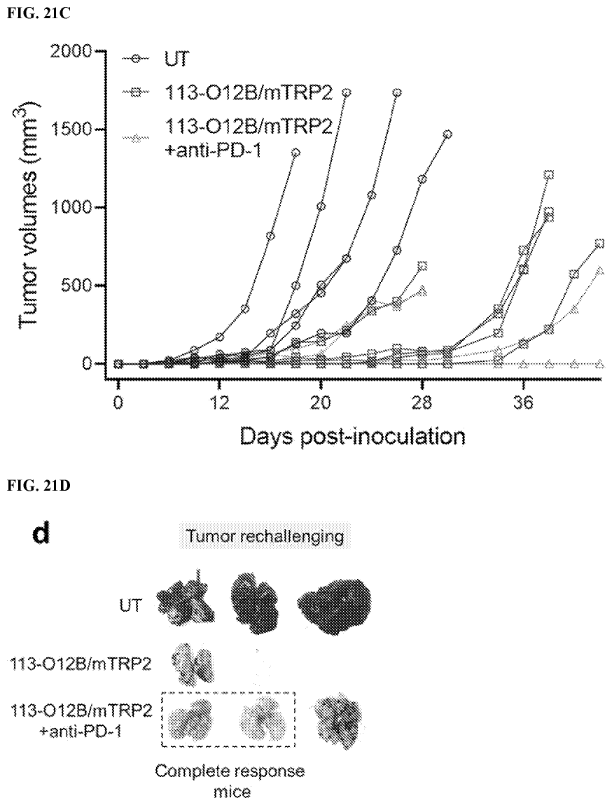

The therapeutic effect of mRNA vaccine to primary melanoma tumor was more critical to the real application in clinic. The gene-engineered tumor B16F10-OVA all expressed OVA antigens, making them easier to be recognized by adaptive immune response generated by mRNA vaccine encoding OVA antigen. However, in primary melanoma tumors, it is difficult to find such a common and specific expressed antigen, so the therapeutic effect to the model based on primary tumor cells faced with more obstacles. Tyrosinase-related protein-2 (TRP2) is a weak tumor-associated antigen expressed in murine and human melanomas. Therefore, induction of strong antibody response and T cell immunity is necessary to generate a strong anti-tumor immunity.²⁰ TRP2₁₈₀₋₁₈₈ peptide (SVYDFFVWL) is MHC class I H-2K^{b} restricted in mouse, which was chosen as the model antigen to evaluate the therapeutic effect of mRNA vaccines.²¹ N¹-methylpseudouridine (N¹mψ) modified TRP2₁₈₀₋₁₈₈ mRNA (mTRP2₁₈₀₋₁₈₈) was synthesized by in vitro transcription (IVT) with 5′ cap and poly A tail (FIG. 21A).

The mice were inoculated with 1 million B16F10 cells at right flank on day 0. Then two group of mice received two doses of mRNA vaccine on day 5 and 12. Anti-PD-1 was also involved in one group on day 7, 11, and 15. First, the percentage of IFN-γ⁺ cells within CD8⁺ T cells stimulated by TRP2₁₈₀₋₁₈₈ peptide was evaluated and shown in FIG. 21B. The vaccination with mRNA vaccine significantly increased the percentage of IFN-γ secreting cells after 1 week of 2^{nd} vaccination. Though the administration with anti-PD-1 didn't make significant difference in the IFN-γ⁺ cells after one week of 2^{nd} vaccination, there were two mice achieved relatively higher responses than the mice treated with 113-O12B/mTRP2₁₈₀₋₁₈₈, suggesting the individual difference of the response to checkpoint inhibition therapy (FIG. 21B). The tumor inhibition was corrected with the T cell response (FIG. 21C). The mice without treatment reached the endpoint within 28 days, while the mRNA vaccine extended the endpoint to more than 36 days. Notably, there were two mice with complete response in the group treated with combination of mRNA vaccine and anti-PD-1, indicating the excellent therapeutic effect of mRNA vaccine in combination with check point inhibitor.

The long-term anti-tumor immunity was also evaluated by the B16F10 metastatic model. Similarly, 1 million B16F10 cells were i.v. injected into the untreated or survived mice on day 30. After 18 days, the lungs were collected and pictured. As shown in FIG. 21D, almost all the lungs of the mice without treatment were occupied by metastatic nodules. Such serious situation was significantly relived in the vaccinated mice. More importantly, no obvious metastatic nodules could be observed in the mice with complete response.

### DISCUSSION

Though the COVID-19 mRNA vaccines exhibited great success in the protection against SARS-COV-2, there was still no cancer vaccine approved in clinic. On the one hand, the weak immunogenicity of tumor antigens required more strong and specific activation of immune system by mRNA vaccine. Second, the undesired expression of tumor antigens in other non-lymphoid organs, such as liver, increased the risk of mRNA vaccine. Targeted delivery of mRNA in lymphoid organs might not only improve the anti-tumor immunity but also reduce the undesired side effects, providing a promising strategy for developing next generation cancer mRNA vaccine. In this work, our group developed a LN-targeting lipid named 113-O12B, which was applied in the therapeutic cancer mRNA vaccine against melanoma. Moreover, the gold standard ALC-0315 used in BNT162b2 was chosen for comparison.

First, the influence of lipid structure on the mRNA expression in LNs was evaluated. The tail length, linker bond, and amine head all showed great impact on the transfection efficacy. The lipids with shorter tail (≤12) exhibited much better efficacy than the longer length (>12). The easter bond linker was better than amide bond linker. Replacing the methyl group in the head amine to other groups also significantly decreased the mRNA expression in LNs. Second, the optimized formulation of 113-O12B/Luc was obtained by changing the weight ratio of different components and replacing the helper lipids, showing better mRNA expression in LNs compared with the gold standard ALC-0315. Notably, ALC-0315/mLuc exhibited very strong signal in liver, while 113-O12B/mLuc delivered mRNA majorly in LNs. The expression of mRNA in liver was also observed in other vaccines by intramuscular injection. The undesired transfection of mRNA in liver might induced side effects of concern, thus 113-O12B showed great advantage in the safety for application in vivo. 113-O12B also promoted better mRNA expression in APCs within LNs compared with ALC-0315, suggesting the superiority of LN-targeting ability. Notably, compared with other targeting systems with the modification of active targeting ligands, 113-O12B exhibited inherent LN targeting ability, which was more convenient for clinical application.

Administration of blank ALC-0315 showed significantly upregulated cytokines and chemokines related to proinflammation, indicating the highly inflammatory of blank ALC-0315. LNPs were reported to be able to induce the inflammatory cell death. Then the inflammatory response induced by blank LNPs might be caused by the damage-associated molecular patterns (DMAPs) released from these cells. The role of the inherent immunogenicity of mRNA or LNPs is still in debate. On the one hand, the immunogenicity activated the innate immune response, benefiting the subsequent activation of adaptive immunity. On the other hand, the high immunogenicity decreased the expression of antigens, thereby hindering the generation of strong adaptive immunity. Whatever, after the encapsulation of OVA mRNA, both the LNP/OVA showed increased secretion of proinflammatory factors, indicating the successful activation of innate and adaptive immunity. The vaccination with both 113-O12B/mOVA and ALC-0315/mOVA showed strong antibody response. Moreover, 113-O12B/OVA elicited stronger CD8⁺ T cell response compared with ALC-0315/mOVA, which still maintained a high level 4 weeks post 2^{nd} vaccination. The vaccination of both LNP/mOVA exhibited full protection of B16F10-OVA tumor over 40 days, confirming the generation of adaptive immunity. Moreover, all the vaccinated mice prevented the growth of metastatic tumor model.

The advantage of LN-targeting 113-O12B/mOVA also benefited the shift of immunocellular composition, which was confirmed by the upregulated infiltration of APCs compared with that of ALC-0315/mOVA. The vaccination by mRNA vaccine reduced the population of Treg cells due to the activation of adaptive immunity. More importantly, the combination of anti-PD-1 significantly decreased the percentage of Treg cells to 2.6%, suggesting the important role of check point inhibitor. the macrophages within the tumor of the vaccinated mice also exhibited the M1 polarization. All these results indicated the vaccination significantly changed the immunocellular composition to inflammatory types.

The therapeutic effect of 113-O12B was evaluated in two kind of model including OVA-engineered B16F10-OVA and original B16F10 tumor model. Tough the vaccination by 113-O12B/mOVA and ALC-0315/mOVA obtained similar T cell response, the tumor inhibition of 113-O12B/mOVA was better than that of ALC-0315/mOVA with a prolonged survival time, which might be induced by the increased infiltration of APCs. The integration of mRNA vaccine and anti-PD-1 antibody eradicated the tumor in 2 of 5 mice. The improved therapeutic outcome was benefited from the activation of cytotoxicity of T cells and the inhibition of Treg cells.

There were two major challenges for the therapeutic effect on original B16F10 tumor model compared with the model antigen OVA engineered cell line, including low immunogenicity of tumor associated antigens and downregulation of the antigens on tumor surface. TRP2₁₈₀₋₁₈₈ peptide was shown to be an effective TAA and was used in this work. Different from the full protein antigen, the LN-targeting delivery of peptide antigens showed more superiority than that of the untargeted ones. The expression of TRP2₁₈₀₋₁₈₈ peptide in APCs could be present to cell surface by directly binding to MHC class I molecules intracellularly after the expression. Then the TRP2₁₈₀₋₁₈₈-specific T cells were activated in LNs by these APCs. Predictively, vaccination with 113-O12B/mTRP2₁₈₀₋₁₈₈ also exhibited excellent antitumor immunity. When combined with anti-PD-1, 113-O12B/mTRP2₁₈₀₋₁₈₈ vaccine showed excellent tumor inhibition with 40% rate of CR. The long-term memory of mRNA vaccine was studied by metastatic tumor model. In all the protection and therapeutic experiments, all the mice with complete response generated no metastatic nodule at all, confirming the long-term efficacy of mRNA vaccine.

In summary, 113-O12B LNP, an inherent LN-targeting LNP delivery system, was identified and applied for cancer mRNA vaccine. The 113-O12B/mRNA showed enhanced expression in APCs compared with that of ALC-0315/mRNA owing to the targeting ability. The vaccination with 113-O12B/mOVA elicited comparable antibody response and CD8⁺ T cell response compared with ALC-0315. Moreover, 113-O12B/mOVA induced more infiltration of APCs to tumor site, leading to improved therapeutic effect on established tumor model compared with ALC-0315. Apart from the full OVA antigen, the mRNA encoding a peptide antigen TRP2₁₈₀₋₁₈₈ was also successfully delivered by 113-O12B, suggesting the ability of processing multiple types of tumor antigens by the LNP/mRNA system. The vaccination with 113-O12B/mTRP2₁₈₀₋₁₈₈ in combination of anti-PD-1 significantly suppressed and even eradicated the established B16F10 tumor. Finally, the mice survived from the therapeutic experiment all showed no growth of metastatic nodules, providing the long-term efficacy of the cancer mRNA vaccine.

### Materials and Methods

### Synthesis and Formulation of LNPs

Generally, LNPs were synthesized by Michael addition of amine-bearing head and acryloyl group containing aliphatic chain. The head and tail were mixed in the molar ratio of 1:4.8 and heated to 70° C. After three days, the mixture was purified by flash chromatography (Combi flash, USA) using silica gel. The lipids were further characterized by electrospray ionization-mass spectrometry before formulation.

The formulated LNPs were prepared by dropwise adding the ethanol solution containing the mixture of active lipid, Chol, helper lipids, and DMG-PEG at the defined weight ratio to 25 mM sodium acetate solution. Then, the mixture was dialyzed with Slide-A-Lyzer™ MINI Dialysis Device (3.5K MWCO, Thermo Scientific, USA). The LNP/mRNA was prepared by simply mixing of blank LNP and mRNA at the weight ratio of 10:1 in phosphate-buffered saline (PBS) solution.

Synthesis of TRP2₁₈₀₋₁₈₈ mRNA

The pMRNA-TRP2₁₈₀₋₁₈₈ plasmid was used as templates for gene polyadenylation using the Tail PCR Primer Mix (System Biosciences, USA), of which reverse primer contains 120 oligodT. The Tail PCR reaction (50 μL) was performed in a Phusion® High-Fidelity DNA Polymerase Kit following the manufacturer's protocol (New England Biolab Inc, USA). The reaction was then applied to a PCR program: 98° C. 3 min, 98° C. 30 s, 64° C. 30 s, 72° C. 10 s, 72° C. 10 min, 4° C. hold. 30 cycles. The PCR mixture was further treated with Proteinase K and purified with a GeneJET PCR Purification Kit (Thermo Scientific, USA). N¹-methylpseudouridine (N¹mψ) modified TRP2₁₈₀₋₁₈₈ mRNA was generated by T7 polymerase through an in vitro transcription reaction. The reaction mixture was treated with DNase I and Antarctic Phosphatase (New England Biolabs, USA) and purified using a MegaClear Kit (Life Technologies, USA). N¹mψ were incorporated to completely substitute the natural counterparts in TRP2₁₈₀₋₁₈₈ mRNA synthesis.

In Vivo Expression of Luc mRNA

BABL/c mice (4-6 weeks old) were injected with LNPs containing 5 μg mRNA and 50 μg active lipid subcutaneously at the tail base. After 6 h of the injection, 100 μL of luciferin at the concentration of 15 mg/mL was injected intraperitoneally. After 10 min, the mice were imaged using the In Vivo Imaging System (IVIS, PerkinElmer). For the thermo stability assay, 113-O12B formulated with Luc mRNA was stored at 4° C. for 0-3 weeks. The IVIS images were taken every week for monitoring the thermo stability of 113-O12B/mLuc.

Delivery of Cre mRNA to LNs in Ai14 Reporter Mice

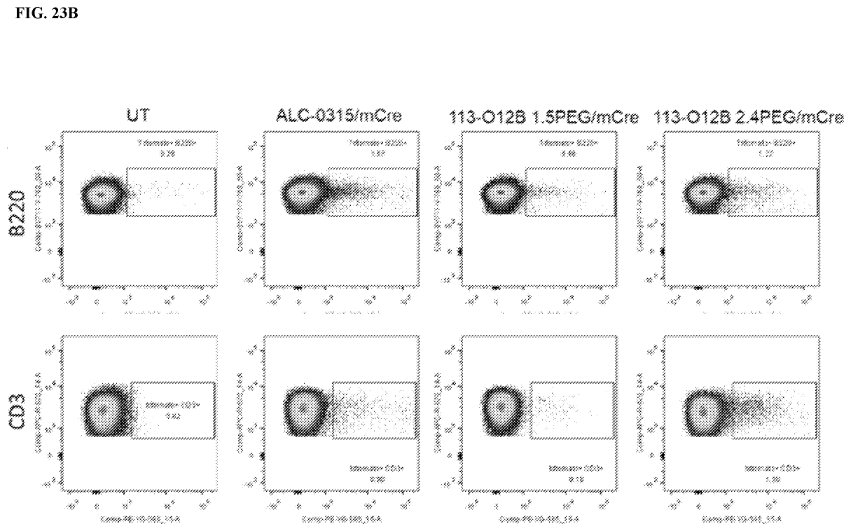

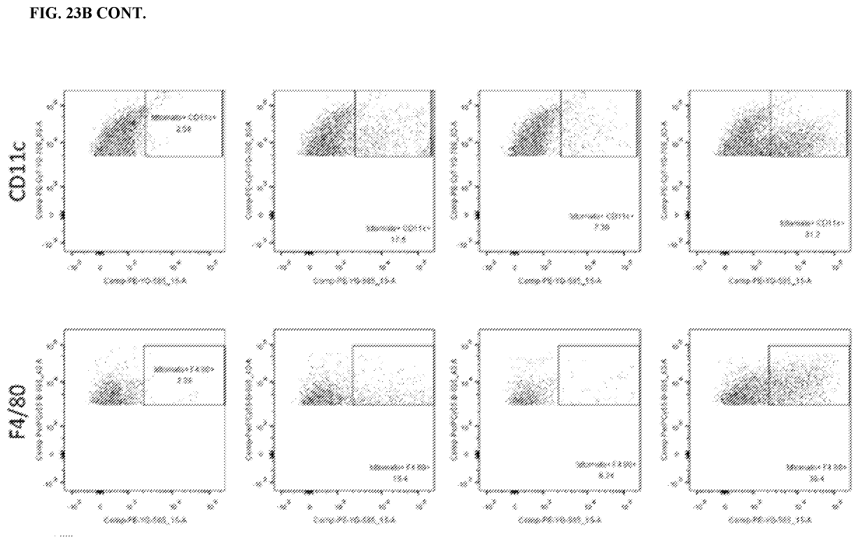

Ai14 mice were injected with different formulation of LNPs/mCre subcutaneously at tail base. After 48 h of the injection, the mice were sacrificed and inguinal LNs were collected. The Cell suspensions were prepared by grinding and filtrating through 70-μm strainer. 2×10⁶ cells were incubated in 100 μL of flow cytometry staining buffer (eBioscience) containing fluorophore-conjugated antibody of interest listed in Table S1 at recommended concentration under 4° C. for 1 h. After washed by staining buffer twice, the cells were kept at 4° C. for analysis. Data was collected by LSR-II flow cytometer (BD Biosciences) and analyzed by Flow Jo-v10. Gating information was shown in FIGS. 23A & 23B.

### ELISA for Antibody Titer

The antibody titer was measured by indirect ELISA assay. The high binding ELISA plates (Greiner Bio-one, USA) were covered with 50 μL of OVA at 20 μg mL⁻¹ in sodium carbonate solution (pH 8.0) at 4° C. overnight. The plates were then washed by PBS with 0.5% tween-20 (PBST) and blocked by 5% bovine serum albumin (BSA) solution (Sigma-Aldrich). The serum collected from immunized mice was diluted in triplicate from 1:100 and then added into the plates for 2 hours at room temperature. Then the plates were washed and incubated with horseradish peroxidase (HRP)-conjugated anti-IgG, IgG1, and IgG2c at 1:10,000 dilution for 1 hours. The plates were washed and incubated with 100 μL of 3,3′,5,5′-tetramethylbenzidine (TMB) substrate (Sigma-Aldrich). The reaction was stopped by 0.16 M sulfuric acid solution. The optical density (O.D.) values at 450 nm was read in BioTex microplate reader. The endpoint titer is defined as the reciprocal of the highest dilution of a serum that gives a reading above the cutoff (4 times of the PBS group).

### Intracellular Cytokine Staining

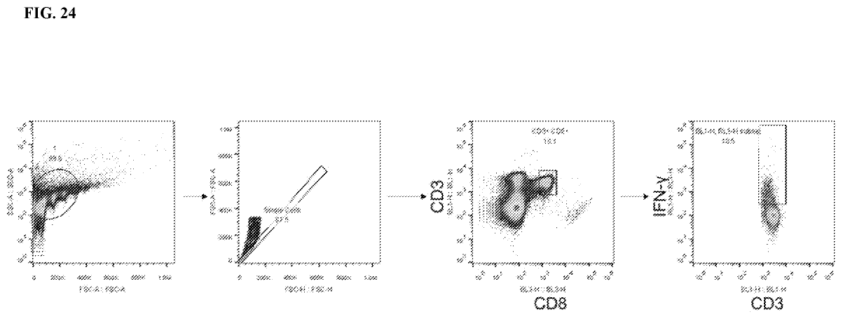

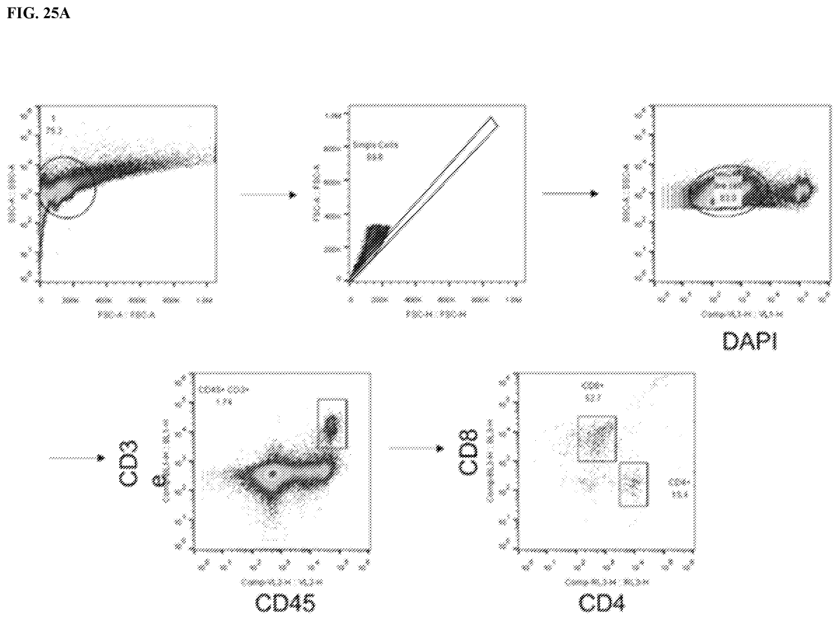

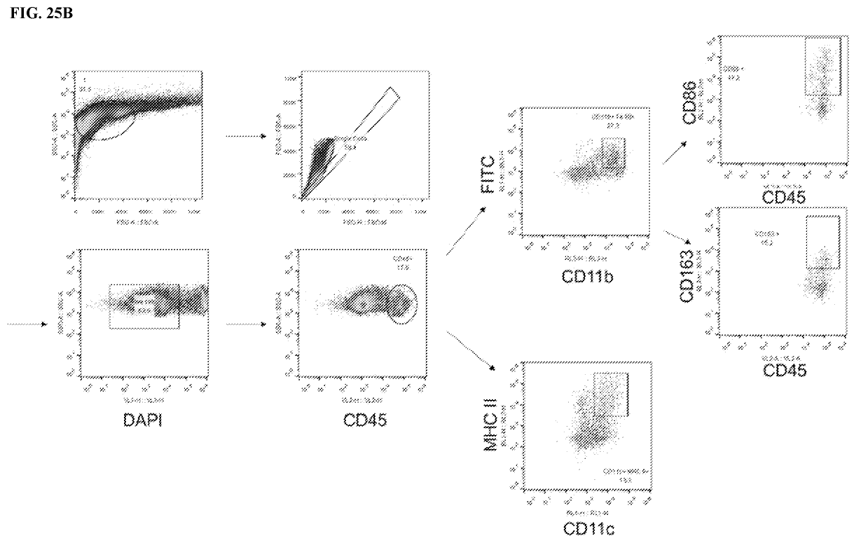

2×10⁶ of spleen cells or peripheral blood mononuclear cells (PBMCs) were isolated and suspended in 200 μL of RPMI-1640 medium containing 10% fetal bovine serum. GolgiPlug™ protein transport inhibitor (BD biosciences) was added to inhibit the intracellular trafficking of cytokines. The cells were the stimulated with respective peptides at 2 ug/mL for 6 h. Then the cells were washed by flow cytometry staining buffer (eBioscience) and then incubated with fluorescent antibodies against surface markers at 4 ºC for 1 h. The cells were washed and fixed by Fixation Buffer (Biolegend) in the dark for 20 minutes at room temperature. The fixed cells were washed with Intracellular Staining Perm Wash Buffer (Biolegend) twice. An optimum concentration of fluorophore-conjugated antibody of interest (e.g., APC anti-IFN-γ and APC anti-FoxP3) for 20 minutes in the dark at room temperature. After washing for another two time, the cells were measured by Attune NxT Flow Cytometer. Data was analyzed by FlowJo-10. Gating information was shown in FIG. 24.

### Tumor Immunocellular Composition Experiment

C57/BL6 mice (4-6 weeks old) were inoculated with 1×10⁶ B16F10-OVA cells at the right flank on day −14. On day 0 and 5, LNPs formulated containing 50 μg active lipid and 5 μg OVA mRNA were injected subcutaneously at tail base as the prime and boost vaccination. Moreover, one of the 113-O12B/mOVA-vaccinated group was treated with anti-PD-1 on day 2 and 7. Tumor was collected on day 12 and suspended using 70 μm cell strainer (Corning, USA). 2×10⁶ cells were stained with fluorophore-conjugated antibody of interest as listed in Table S1 at 4 ºC for 1 h. Then the cells were washed and analyzed by by Attune NxT Flow Cytometer. Gating information was shown in FIG. 24.

### ELISpot Assay

After 1 week of the 2^{nd} vaccination, the mice (n=5) were sacrificed, and the spleen was collected and made into cell suspensions using 70 μm cell strainer. The ELISpot assay was conducted using the Mouse Interferon gamma ELISPOT Kit (ab64029, Abcam, USA). 2×10⁴ of spleen cells were incubated in complete RPMI-1640 medium with or without 2 ug/mL of SIINFEKL peptide at 37° C. for 12 h. Then the plates were washed, incubated with biotinylated anti-IFN-γ antibody, and then streptavidin-alkaline phosphatase conjugate according to the manufacturer's protocol. The pictures were taken, and spot numbers of each mouse were calculated automatically.

### Immunization and Tumor Therapy

For OVA engineered B16F10-OVA tumor model, 1×10⁶ B16F10-OVA cells were injected subcutaneously at the right flank of C57/BL6 mice (4-6 weeks old, n=5)) on day 0. The mice received two doses of vaccination at the dose equivalent to 50 μg active lipid and 5 μg OVA mRNA on day 5 and 12. The control mice were without any treatment. For one group vaccinated by 113-O12B/mOVA, the mice were also treated with anti-PD-1 on day 7, 11, and 15. The length (L) and width (W) of the tumors were measure every other day and the tumor volumes (V) were calculated by the equation: V=L×W²/2. On day 30, the mice without obvious tumors were rechallenging with 1×10⁶ B16F10-OVA cells by i.v. injection. A control group (n=5) was also treated with 1×10⁶ B16F10-OVA cells by i.v. injection. On day 48, all the mice were sacrificed, and the lungs were collected for photograph. For original B16F10 tumor model, all the protocols were similar to those of B16F10-OVA model except the does, which was equivalent to 30 μg active lipid and 3 μg TRP2₁₈₀₋₁₈₈ mRNA.

TABLE S1
Antibody used for flow cytometry

| Target | Label | Company | Catalog |
|---|---|---|---|
| CD3e | APC | eBioscience ™ | 50-112-9569 |
| B220 | Brilliant Violet 650 | BD biosciences | BDB563893 |
| NK-1.1 | APC-Cy7 | BD biosciences | BDB560618 |
| CD11c | PE-Cyanine7 | eBioscience | 50-154-55 |
| F4/80 | PerCP-Cyanine5.5 | eBioscience | 50-112-9034 |
| IFN-γ | APC | Biolegend | 50-169-921 |
| CD8a | PE-Cy5 | BD biosciences | BDB561094 |
| CD4 | APC-Cy7 | BD biosciences | BDB561830 |
| CD3 | FITC | eBioscience | 50-112-9706 |
| FoxP3 | APC | R&D system | FPK8214A025 |
| CD45 | Brilliant Violet 510 | BD biosciences | BDB563891 |
| MHC Class II | PE | eBioscience | 50-108-18 |
| CD11b | APC/Cy7 | Biolegend | 50-162-532 |
| CD163 | PE/Cyanine7 | Biolegend | 155319 |
| CD86 | PE | BD biosciences | BDB561129 |

TABLE 1
SEQUENCES
Sequences used for
5′UTRs optimization in
pcDNA3.0-eGFP and pMRNA-Fluc-eGFP.

| Name | Sequence^{α} |
|---|---|
| S27a-45′ | CCACTAGTTCTAGAGGTACC |
| 5′ UTR | TTGGACCCTCGTACAGAAGC |
| | TAATACGACTCACTATAGGG |
| | AGGAAAGAATCGCATCGGCT |
| | GTATAAGAAAGCCTTTTGAG |
| | GCATTTTTTTTAGTTGAGCA |
| | CATCATTTCGAGGCCATTCT |
| | GAGGTAAACCGAGAAAAGAG |
| | CGTAAAGAAACCGAGCGAAC |
| | GAGCAAATCTGGCACTGCGT |
| | TAGACAGCCGCGATTCCGCT |
| | GCAGCGCGCAGGCACGTGTG |
| | TGGCCGCCTAAGGGGGGGGT |
| | CCTTCGGCCAGGAGACCCCG |
| | TCGGCCACGCTCGGATCTTC |
| | CTTTCGGATCCGCCATCGTG |
| | GGTGGAGCCGCCGCCACGGA |
| | ATTCGCCACCATGGAAGA |
| NGA-7d | CCACTAGTTCTAGAGGTACC |
| | TTGGACCCTCGTACAGAAGC |
| | TAATACGACTCACTATAGGC |
| | AAAAATCAAAATCAATCATC |
| | ATCACAACATCAACAATCAA |
| | TCATCAACACATCATCAAGA |
| | CACCACCGAATTCGCCACCA |
| | TGGAAGA |
| 70nt | CCACTAGTTCTAGAGGTACC |
| | TTGGACCCTCGTACAGAAGC |
| | TAATACGACTCACTATAGGG |
| | AAGAGATAAACATAAACATA |
| | AACGACAAGAAACACATACA |
| | AAAGAAACAGGACAGAAAAC |
| | AGCCACCGAATTCGCCACCA |
| | TGGAAGA |
| Ces1d | CCACTAGTTCTAGAGGTACC |
| | TTGGACCCTCGTACAGAAGC |
| | TAATACGACTCACTATAAGG |
| | AGGCGGGTCCCCTGGTCCAC |
| | AACAGAAGCATTGCTAAAGC |
| | AGCAGATAGCTCAGAGACCC |
| | ACAGAGCCCTTGTCCTTCCA |
| | CAGAATTCGCCACCATGGAA |
| | GA |

^{α}Homolog arms with pMRNA-FLuc-eGFP were underlined. Restriction enzyme cutting sites were bolded.

TABLE 2
Sequences used for 3′UTRs optimization in
pcDNA3.0-eGFP and pMRNA-Fluc-eGFP

| Name | Sequence |
|---|---|
| S107_XbaI-Moderna | AGCTGTACAAGTAATCTAGA |
| 3′UTR-ApaI | GCTGGAGCCTCGGTGGCCTA |
| | GCTTCTTGCCCCTTGGGCCT |
| | CCCCCCAGCCCCTCCTCCCC |
| | TTCCTGCACCCGTACCCCCG |
| | TGGTCTTTGAATAAAGTCTG |
| | AGTGGGGGGCAGGGCCCTAT |
| | TCTATAGTGTC |
| S108_XbaI-Pfizer | AGCTGTACAAGTAATCTAGA |
| 3′UTR-ApaI | CTCGAGCTGGTACTGCATGC |
| | ACGCAATGCTAGCTGCCCCT |
| | TTCCCGTCCTGGGTACCCCG |
| | AGTCTCCCCCGACCTCGGGT |
| | CCCAGGTATGCTCCCACCTC |
| | CACCTGCCCCACTCACCACC |
| | TCTGCTAGTTCCAGACACCT |
| | CCCAAGCACGCAGCAATGCA |
| | GCTCAAAACGCTTAGCCTAG |
| | CCACACCCCCACGGGAAACA |
| | GCAGTGATTAACCTTTAGCA |
| | ATAAACGAAAGTTTAACTAA |
| | GCTATACTAACCCCAGGGTT |
| | GGTCAATTTCGTGCCAGCCA |
| | CACCCTGGAGCTAGCGGGCC |
| | CTATTCTATAGTGTC |
| S109_XbaI- | AGCTGTACAAGTAATCTAGA |
| Apolipoprotein | AGTGTCCAGACCATTGTCTT |
| A-II 3′UTR-ApaI | CCAACCCCAGCTGGCCTCTA |
| | GAACACCCACTGGCCAGTCC |
| | TAGAGCTCCTGTCCCTACCC |
| | ACTCTTTGCTACAATAAATG |
| | CTGAATGAATCCAGGGCCCT |
| | ATTCTATAGTGTC |
| S110_XbaI-Cytochrome | AGCTGTACAAGTAATCTAGA |
| P450 | GTGTGTGGAGGACACCCTGA |
| 2E1 3′UTR-ApaI | ACCCCCCGCTTTCAAACAAG |
| | TTTTCAAATTGTTTGAGGTC |
| | AGGATTTCTCAAACTGATTC |
| | CTTTETTTGCATATGAGTAT |
| | TTGAAAATAAATATTTTCCC |
| | AGAATATAAATAAATCATCA |
| | CATGATTATTTTAACTATGG |
| | GCCCTATTCTATAGTGTC |
| S111_XbaI-Complement | GTCTGGAGTTCTTTGCCAAG |
| Component 3 | AGGGAGAGGCTGAAATCCCC |
| 3′UTR-ApaI | AGCCGCCTCACCTGCAGCTC |
| | AGCTCCATCAGCTGTACAAG |
| | TAATCTAGACCACACCCCCA |
| | TTCCCCCACTCCAGATAAAG |
| | CTTCAGTTATATCTCACGTC |
| | TACTTGAAACCTCACCTGTT |
| | CCCACCGCATTTTCTCCTGG |
| | CGTTCGCCTGCTAGTGTGGG |
| | GCCCTATTCTATAGTGTC |
| S112_XbaI-YY2 | AGCTGTACAAGTAATCTAGA |
| Transcription | GTATTTTCCTGCTTAAAAAA |
| Factor 3′UTR-ApaI | GTTATATAGGTGTTATTTGT |
| | TTTAATCTTGGTTGTAGTCT |
| | TGGATGTTAACACATCTTGC |
| | ATTTTAGCTGTATTAGGTCA |
| | TGTAGTATTGATATTAGGTG |
| | ATTTAATAGTACTAGTTTAA |
| | ACCTATTTTAGTCATTTTAT |
| | GGGCCCTATTCTATAGTGTC |
| S113_XbaI-TIAM1 | AGCTGTACAAGTAATCTAGA |
| 3′UTR-ApaI | CGCCTTTCGGGTTTCCAGGG |
| | CTTCGAGCTTGATCTTTTGA |
| | AAGTTTTATTCTATTAAATT |
| | TTTGCTATATCTTCTGGTTT |
| | TCTGAAAAAGCTTTAGAATG |
| | GTTTCTATACCCTTTGTATC |
| | ACTGCATTTTTCCATATCAT |
| | CTCCGGTTCGATCGCGTCCA |
| | GGGCCCTATTCTATAGTGTC |
| S114_XbaI-FAM171A1 | AGCTGTACAAGTAATCTAGA |
| 3′UTR-ApaI | AAGCTGTCGTTGAGAACTTA |
| | GGTTGGCACGTAGCGTCTCA |
| | AGGTATGCGTTCTCTCAAAG |
| | GAAAGCTATGCATCGCTGCT |
| | TCGTTGTCTGATTTTGCTTA |
| | GATTTTGCTTTGGTTAGGTT |
| | GCGTTTTGGGGTTTGCCYTT |
| | TTTTGTTGTCGCTTAAATGC |
| | GGGCCCTATTCTATAGTGTC |
| S115_XbaI-AP381 | AGCTGTACAAGTAATCTAGA |
| 3′UTR-ApaI | AGAATGCTAGTGTGTATCTA |
| | TCATGTATGCAATACTTTCC |
| | CCCTTTTTGCTTTGCTAACC |
| | AAAGAGCATATATTTTACTG |
| | TCAGTTGTCTCAACTCTTGA |
| | ATCCATGTGGCGTTTTCTCT |
| | GTCCTGCTGCTTCTTTTGGC |
| | CTCCTCGTTTTCCTTCTCTT |
| | GGGCCCTATTCTATAGTGTC |
| S116_XbaI-OXR1 | AGCTGTACAAGTAATCTAGA |
| 3′UTR-ApaI | GCAATACAGTGTAACATGTC |
| | ACTTGTGCTTTAAAATTAGT |
| | CTGTATCACCATTTATTACA |
| | GTTATAATTTTGGAGTTTAT |
| | TTTTCAAATCATGTTCTTGT |
| | CCCAGAGTTCTTTAGGTTAA |
| | CACTAGGGACTGCGTCCATG |
| | TACTAGTATAACAGCTTGGG |
| | GGGCCCTATTCTATAGTGTC |
| S117_XbaI-POTEE | AGCTGTACAAGTAATCTAGA |
| 3′UTR-ApaI | TGGCCCAGTCCTCTCCCTAG |
| | TTCACACAGGGGAGGTGATA |
| | GCATTGCTTTTGTGCAAATT |
| | ACATAATGCAAAATTTTTTG |
| | AATCTTCGCCTTAATACTTT |
| | TTAATTTTGTTTTATTTTGA |
| | ATGATCAGCCTTCGTGGCCC |
| | CCCTCTTTTGTACCCCAACT |
| | GGGCCCTATTCTATAGTGTC |
| S118_XbaI-WIP12 | AGCTGTACAAGTAATCTAGA |
| 3′UTR-ApaI | ATCCTGCTTATGAATTTTAG |
| | CTTTTTGTTTGTTTGTTTTC |
| | TCTTTTTGCCAAAATTAACT |
| | GTTTGGTGAAGCCCGCAAAA |
| | CCTCCTCGCTTTGCATGCAT |
| | GAACGTGCCAAGCCAGCATA |
| | GGGGAGCTAGAAGCCACTTT |
| | CCAGCCACCTGCCGTTGGGT |
| | GGGCCCTATTCTATAGTGTC |
| S119_XbaI-50_M_ | TGAAAAATAAGATACTGTGG |
| T1012 3′UTR-ApaI | ATATCTAACCAATCGCACTT |
| | AACGACTCGGGCCACCACTG |
| | TCGGGCCCAGCTGTACAAGT |
| | AATCTAGACGACTATTTAAA |
| | CAGACTGTTTTAATCTGTGA |
| | AAATGGTTGGTAACTGTATT |
| | CTATAGTGTC |

^{α}Homolog arms with pcDNA3.e-eGFP were underlined.

TABLE 3
All primers used in plasmids construction

| Name | Sequence |
|---|---|
| A93_pMRNA_SUTR_ | ggTTGGACCCTCGTACAGAAGCTAA |
| Forward | |
| A95_pMRNA pA_ | TTTTTTTTTTTTTTTTTTTTTTTTT |
| Reverse | TTTTTTTTTTTTTTTTTTTTTTTTT |
| | TTTTTTTTTTTTTTTTTTTTTTTTT |
| | TTTTTTTTTTTTTTTTTTTTTTTTT |
| | TTTTTTTTTTTTTTTTTTTTTT |
| AZ04_Original | GAATTCGGGAAATAAGAG |
| 5′UTR_F | |
| A205_Original | GAATTCTCTTATATTTCTTC |
| 5′UTR_R | |
| A206_S27a-45′ | GAATTCGGGAGGAAAGA |
| 5′ UTR_F | |
| A207_S27a-45′ | GAATTCCGTGGGGGGGG |
| 5′ UTR_R | |
| A208_S27a-44′ | GAATTCGGGGATCOGCCA |
| 5′UTR_F | |
| A209_S27a-44′ | GAATTCCGTGGGGGGGG |
| 5′UTR_R | |
| A210_NCA-7d_F | GAATTCGGCAAAAATCAAA |
| A211_NCA-7d_R | GAATTCGGTGGTGTCTTGA |
| A212_70 nt_F | GÅATTCGGGAAGAGATAAA |
| A213 70 nt_R | GAATTCGGTGGCTGTTTTC |
| A214 Ces1d_F | GAATTCAGGAGGCGGGTC |
| A215 Ces1d_R | GAATTCTGTGGAAGGACAAG |
| A216_KpnI-T7-527a-45′ | CCACTAGTTCTAGAGGTACC |
| 5′ UTR-EcoRI_F | |
| A217_KpnI-T7-527a-45′ | GAATTCCGTGGGGGGGG |
| 5′ UTR-EcoRI_R | |
| A218_KpnI-T7-527a-44′ | CCACTAGTTCTAGAGGTACC |
| 5′ UTR-EcoRI_F | |
| A219_KpnI-T7-627a-44′ | GAATTCCGTGGCGGCGG |
| 5′ UTR-EcoRI_R | |
| A220_KpnI-T7-NCA-7d | CCACTAGTTCTAGAGGTACC |
| 5′ UTR-EcoRI_F | |
| A221_Kpn/-T7-NCA-7d | TCTTCCATGGTGGCGAATTC |
| 5′ UTR-EcoRI_R | |
| A22Z_KpnI-T7- | CCACTAGTTCTAGAGGTACC |
| Ces1d-5′ UTR_F | |
| A223_KpnI-17- | TCTTCCATGGTGGCGAATTC |
| Ces1d-5′ UTR_R | |
| A224_KpnI-17- | CCACTAGTICTAGAGGTACC |
| 70 nt-5′ UTR_F | |
| A225_KpnI-T7- | TCTTCCATGGTGGCGAATTC |
| 70 nt-5′ UTR_R | |
| A333_pcDNAeGFP_ | AGCTGTACAAGTAATCTAGA |
| 3′UTR_XbaI_F | |
| A334_pcDNAeGFP_ | GACACTATAGAATAGGGCC |
| 3′UTR_ApaI_R | |
| A348_pMRNA_Ces1d_ | CCactagttctagaggtacc |
| Fluc eGFP_KpnI_F | |
| A349_pMRNA_Ces1d_ | ttacttgtacagctcgtcca |
| Fluc_eGFP_R | |
| A350_pMRNA 3′UTR_F | AGCTGTACAAGTAATCTAGA |
| A351_pMRNA_3′UTR_ | ttttttttttttttsatatt |
| Sspl_R | GACACTATAGAATA |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A nanoparticle, comprising a plurality of lipidoids; and an adjuvant, an antigen, or a nucleic acid, wherein each lipidoid has a structure represented by formula I: 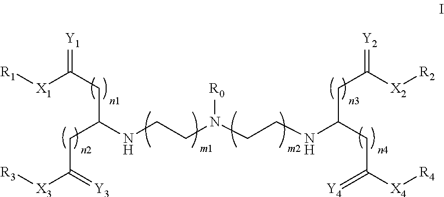
or a pharmaceutically acceptable salt thereof,
wherein,
each Y₁, Y₂, Y₃, Y₄, X₁, X₂, X₃, and X₄ is independently selected from the group consisting of O, S, and NR₅;
R₀ is H or alkyl;
R₁, R₂, R₃, and R₄ are each independently selected from the group consisting of alkyl, alkyloxyalkyl, alkylaminoalkyl, alkylthioalkyl, alklydisulphidealkyl, alkylamidoalkyl, alkylesteralkyl, alkylcarbamatealkyl, alkylcarbonatealkyl, and alkylurealylalkyl;
each R₅ is independently selected from hydrogen, alkyl, or aralkyl;
n1, n2, n3, and n4 are each independently 1, 2, 3, 4, 5, 6, 7, 8, 9, or 10; and
m1 and m2 are each independently 1, 2, 3 4, or 5.

2. The nanoparticle of claim 1, wherein each lipidoid has structure represented by formula I.

3. The nanoparticle of claim 1 or 2, wherein R₀ is methyl or ethyl.

4. The nanoparticle of any one of claims 1-3, wherein R₀ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

5. The nanoparticle of any one of claims 1-4, wherein R₀ is substituted with hydroxyl.

6. The nanoparticle of any one of claims 1-5, wherein Y₁ is O.

7. The nanoparticle of any one of claims 1-6, wherein Y₂ is O.

8. The nanoparticle of any one of claims 1-7, wherein Y₃ is O.

9. The nanoparticle of any one of claims 1-8, wherein Y₄ is O.

10. The nanoparticle of any one of claims 1-9, wherein X₁ is O.

11. The nanoparticle of any one of claims 1-9, wherein X₁ is NH.

12. The nanoparticle of any one of claims 1-11, wherein X₂ is O.

13. The nanoparticle of any one of claims 1-11, wherein X₂ is NH.

14. The nanoparticle of any one of claims 1-13, wherein X₃ is O.

15. The nanoparticle of any one of claims 1-13, wherein X₃ is NH.

16. The nanoparticle of any one of claims 1-15, wherein X₄ is O.

17. The nanoparticle of any one of claims 1-15, wherein X₄ is NH.

18. The nanoparticle of any one of claims 1-17, wherein n1 is 2.

19. The nanoparticle of any one of claims 1-18, wherein n2 is 2.

20. The nanoparticle of any one of claims 1-19, wherein n3 is 2.

21. The nanoparticle of any one of claims 1-20, wherein n4 is 2.

22. The nanoparticle of any one of claims 1-21, wherein m1 is 1.

23. The nanoparticle of any one of claims 1-22, wherein m2 is 1.

24. The nanoparticle of claim 1 or 2, wherein each lipidoid has a structure represented by formula Ia: 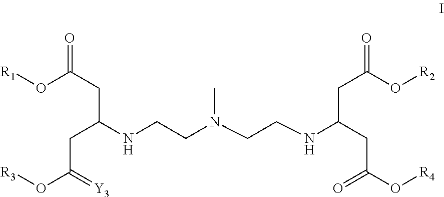
or a pharmaceutically acceptable salt thereof.

25. The nanoparticle of any one of claims 1-24, wherein R₁ is alkyloxyalkyl.

26. The nanoparticle of any one of claims 1-24, wherein R₁ is alkylthioalkyl.

27. The nanoparticle of any one of claims 1-24, wherein R₁ is alklydisulphidealkyl.

28. The nanoparticle of any one of claims 1-27, wherein R₂ is alkyloxyalky.

29. The nanoparticle of any one of claims 1-27, wherein R₂ is alkylthioalkyl.

30. The nanoparticle of any one of claims 1-27, wherein R₂ is alklydisulphidealkyl.

31. The nanoparticle of any one of claims 1-30, wherein R₃ is alkyloxyalky.

32. The nanoparticle of any one of claims 1-30, wherein R₃ is alkylthioalkyl.

33. The nanoparticle of any one of claims 1-30, wherein R₃ is alklydisulphidealkyl.

34. The nanoparticle of any one of claims 1-33, wherein R₄ is alkyloxyalky.

35. The nanoparticle of any one of claims 1-33, wherein R₄ is alkylthioalkyl.

36. The nanoparticle of any one of claims 1-33, wherein R₄ is alklydisulphidealkyl.

37. The nanoparticle of any one of claims 1-36, wherein R₁ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

38. The nanoparticle of any one of claims 1-37, wherein R₂ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

39. The nanoparticle of any one of claims 1-38, wherein R₃ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

40. The nanoparticle of any one of claims 1-39, wherein R₄ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

41. The nanoparticle of any one of claims 1-24, wherein R₁ has a structure represented by formula IIa: 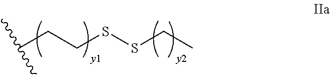
wherein,
y1 is 1, 2, 3, 4 or 5;
y2 is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
the wavy line indicates a connection to X₁.

42. The nanoparticle of claim 41, wherein y1 is 1.

43. The nanoparticle of claim 41 or 42, wherein y2 is 5.

44. The nanoparticle of claim 41 or 42, wherein y2 is 7.

45. The nanoparticle of claim 41 or 42, wherein y2 is 9.

46. The nanoparticle of claim 41 or 42, wherein y2 is 11.

47. The nanoparticle of claim 41 or 42, wherein y2 is 13.

48. The nanoparticle of any one of claims 1-24 and 41-47, wherein R₂ has a structure represented by formula IIb: 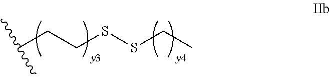
wherein,
y3 is 1, 2, 3, 4 or 5;
y4 is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
the wavy line indicates a connection to X₂.

49. The nanoparticle of claim 48, wherein y3 is 1.

50. The nanoparticle of claim 48 or 49, wherein y4 is 5.

51. The nanoparticle of claim 48 or 49, wherein y4 is 7.

52. The nanoparticle of claim 48 or 49, wherein y4 is 9.

53. The nanoparticle of claim 48 or 49, wherein y4 is 11.

54. The nanoparticle of claim 48 or 49, wherein y4 is 13.

55. The nanoparticle of any one of claims 1-24 and 41-54, wherein R₃ has a structure represented by formula IIc: 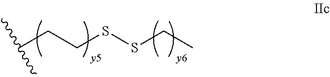
wherein,
y5 is 1, 2, 3, 4 or 5;
y6 is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
the wavy line indicates a connection to X₃.

56. The nanoparticle of claim 55, wherein y5 is 1.

57. The nanoparticle of claim 55 or 56, wherein y6 is 5.

58. The nanoparticle of claim 55 or 56, wherein y6 is 7.

59. The nanoparticle of claim 55 or 56, wherein y6 is 9.

60. The nanoparticle of claim 55 or 56, wherein y6 is 11.

61. The nanoparticle of claim 55 or 56, wherein y6 is 13.

62. The nanoparticle of any one of claims 1-24 and 41-61, wherein Ra has a structure represented by formula IId: 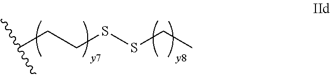
wherein,
y7 is 1, 2, 3, 4 or 5;
y8 is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
the wavy line indicates a connection to X₄.

63. The nanoparticle of claim 62, wherein y7 is 1.

64. The nanoparticle of claim 62 or 63, wherein y8 is 5.

65. The nanoparticle of claim 62 or 63, wherein y8 is 7.

66. The nanoparticle of claim 62 or 63, wherein y8 is 9.

67. The nanoparticle of claim 62 or 63, wherein y8 is 11.

68. The nanoparticle of claim 62 or 63, wherein y8 is 13.

69. The nanoparticle of any one of claims 1-68, wherein the plurality of lipidoids forms a bilayer.

70. The nanoparticle of any one of claims 1-69, wherein the nanoparticle comprises an adjuvant.

71. The nanoparticle of claim 70, wherein the adjuvant is a stimulator of the immune system.

72. The nanoparticle of claim 71, wherein the stimulator of the immune system stimulates innate immunity.

73. The nanoparticle of claim 71, wherein the stimulator of the immune system is a stimulator of interferon genes (STING).

74. The nanoparticle of claim 73, wherein the STING is a STING agonist.

75. The nanoparticle of claim 74, wherein the STING agonist is a cyclic dinucleotide.

76. The nanoparticle of claim 75, wherein the STING agonist is cyclic guanosine monophosphate-adenosine monophosphate (cGAMP).

77. The nanoparticle of any one of claims 1-76, wherein the adjuvant is encapsulated within the nanoparticle.

78. The nanoparticle of any one of claims 69-77, wherein the plurality of lipidoids forms a bilayer and the adjuvant is encapsulated within the bilayer.

79. The nanoparticle of any one of claims 1-69, wherein the nanoparticle comprises an antigen.

80. The nanoparticle of claim 79, wherein the antigen is a vaccine.

81. The nanoparticle of claim 79 or 80, wherein the antigen is a protein.

82. The nanoparticle of claim 79 or 80, wherein the antigen is an attenuated virus.

83. The nanoparticle of any one of claims 79-82, wherein the antigen is encapsulated within the nanoparticle.

84. The nanoparticle of any one of claims 79-83, wherein the plurality of lipidoids forms a bilayer and the antigen is encapsulated within the bilayer.

85. The nanoparticle of any one of claims 1-69, wherein the nanoparticle comprises a nucleic acid.

86. The nanoparticle of claim 85, wherein the nucleic acid is a DNA or a RNA.

87. The nanoparticle of claim 85, wherein the nucleic acid is an RNA.

88. The nanoparticle of claim 87, wherein the RNA is an mRNA.

89. The nanoparticle of claim 88, wherein when the mRNA contacts a cell, the mRNA induces the synthesis of a protein belonging to a cancer cell.

90. The nanoparticle of claim 89, wherein the cancer cell is a bladder cancer cell, breast cancer cell, brain cancer cell, bone cancer cell, cervical cancer cell, colorectal cancer cell, head cancer cell, neck cancer cell, kidney cancer cell, liver cancer cell, lung cancer cell, lymphoma cell, mesothelioma cell, myeloma cell, prostate cancer cell, skin cancer cell, thyroid cancer cell, ovarian cancer cell, or uterine cancer cell.

91. The nanoparticle of claim 88, wherein, when the mRNA contacts a cell, the mRNA induces the synthesis of a protein belonging to a virus.

92. The nanoparticle of claim 91, wherein the virus is hepatitis C, norovirus, junin, dengue virus, coronavirus, human immunodeficiency virus, herpes simplex, avian flu, chickenpox, cold sores, common cold, glandular fever, influenza, measles, mumps, pharyngitis, pneumonia, rubella, severe acute respiratory syndrome, and lower or upper respiratory tract infection (e.g., respiratory syncytial virus).

93. The nanoparticle of claim 92, wherein the virus is an influenza virus.

94. The nanoparticle of claim 92, wherein the virus is a human immunodeficiency virus.

95. The nanoparticle of claim 92, wherein the virus is a coronavirus.

96. The nanoparticle of claim 95, wherein the coronavirus is SARS-COV-2.

97. The nanoparticle of claim 96, wherein the SARS-COV-2 is the alpha, beta, gamma, delta, omicron, or BA.2 strain of SARS-COV-2.

98. The nanoparticle of claim 66 or 67, wherein when the mRNA contacts a cell, the mRNA induces the synthesis of the spike protein of the SARS-COV-2.

99. The nanoparticle of claim 85, wherein the nucleic acid has at least 60%, 65%, 70%, 75%, 80%, 85%, 90%, 95%, or 99% sequence identity to any one of the sequences recited in Sequences—Table 1, Sequences—Table 2, or Sequences—Table 3.

100. The nanoparticle of claim 85, wherein the nucleic acid has at least 75%, 80%, 85%, 90%, 95%, or 99% sequence identity to any one of the sequences recited in Sequences—Table 1, Sequences—Table 2, or Sequences—Table 3.

101. The nanoparticle of claim 85, wherein the nucleic acid has at least 90%, 95%, or 99% sequence identity to any one of the sequences recited in Sequences—Table 1, Sequences—Table 2, or Sequences—Table 3.

102. The nanoparticle of claim 85, wherein the nucleic acid has at least 95%, or 99% sequence identity to any one of the sequences recited in Sequences—Table 1, Sequences Table 2, or Sequences—Table 3.

103. The nanoparticle of claim 85, wherein the nucleic acid has a sequence according to any one of the sequences recited in Sequences—Table 1, Sequences—Table 2, or Sequences—Table 3.

104. The nanoparticle of any one of claims 85-103, wherein the nucleic acid is encapsulated within the nanoparticle.

105. The nanoparticle of any one of claims 85-104, wherein the plurality of lipidoids forms a bilayer and the nucleic acid is encapsulated within the bilayer.

106. The nanoparticle of any one of claims 1-105, wherein the nanoparticle further comprises a chemotherapeutic agent.

107. The nanoparticle of claim 106, wherein the chemotherapeutic agent is cytotoxic.

108. The nanoparticle of claim 106 or 107, wherein the chemotherapeutic agent is an alkylating agent, an antimetabolite, an anti-microtubule agent, anti-tumor anti-biotic, or a topoisomerase inhibitor, a mitotic inhibitor, or a corticosteroid.

109. The nanoparticle of any one of claims 106-108, wherein the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat.

110. The nanoparticle of claim 106, wherein the chemotherapeutic agent is doxorubicin.

111. The nanoparticle of any one of claims 106-110, wherein the chemotherapeutic agent is encapsulated within the nanoparticle.

112. The nanoparticle of any one of claims 1-111, wherein the nanoparticles are capable of internalizing an antigen (e.g., an antigen from a cancer cell).

113. The nanoparticle of any one of claims 1-112, wherein the nanoparticle has a diameter of 25-500 nm.

114. The nanoparticle of any one of claims 1-112, wherein the nanoparticle has a diameter of 50-250 nm.

115. The nanoparticle of any one of claims 1-112, wherein the nanoparticle has a diameter of about 50 nm, about 75 nm, about 100 nm, about 125 nm, about 150 nm, about 175 nm, about 200 nm, about 225 nm, or about 250 nm.

116. The nanoparticle of any one of claims 1-112, wherein the nanoparticle has a diameter of about 50 nm, about 75 nm, about 100 nm, about 125 nm, about 150 nm, about 175 nm, or about 200 nm.

117. The nanoparticle of any one of claims 1-116, wherein the nanoparticle has a pKa of 7-8.

118. The nanoparticle of any one of claims 1-116, wherein the nanoparticle has a pKa of about 7.0, about 7.1, about 7.2, about 7.3, about 7.4, about 7.5, about 7.6, about 7.7, about 7.8, about 7.9, or about 8.0.

119. The nanoparticle of any one of claims 1-116, wherein the nanoparticle has a pKa of about 7.3, about 7.4, about 7.5, about 7.6, or about 7.7.

120. A pharmaceutical composition comprising a nanoparticle of any one of claims 1-119 and a pharmaceutically acceptable excipient.

121. A method of treating cancer in a subject in need thereof comprising administering a therapeutically effective amount of the nanoparticle of any one of claims 1-119 or a pharmaceutically acceptable salt thereof to the subject.

122. A method of treating cancer in a subject in need thereof comprising the steps of administering a therapeutically effective amount of a chemotherapeutic agent to the subject; and
administering a therapeutically effective amount of the nanoparticle of claim 1-119 to the subject.

123. The method of claim 122, wherein the chemotherapeutic agent is cytotoxic.

124. The method of claim 122 or 123, wherein the chemotherapeutic agent is an alkylating agent, an antimetabolite, an anti-microtubule agent, anti-tumor antibiotic, or a topoisomerase inhibitor, a mitotic inhibitor, or a corticosteroid.

125. The method of claim 124, wherein the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat.

126. The method of claim 124, wherein the chemotherapeutic agent is doxorubicin.

127. The method of any one of claims 121-126, wherein the nanoparticle is administered intratumorally.

128. The method of any one of claims 121-126, wherein the chemotherapeutic is administered intratumorally.

129. The method of any one of claims 122-128, wherein the nanoparticle is administered about 6-48 hours after the chemotherapeutic agent.

130. The method of any one of claims 122-128, wherein the nanoparticle is administered about 6-24 hours after the chemotherapeutic agent.

131. The method of any one of claims 122-128, wherein the nanoparticle is administered about 24 hours after the chemotherapeutic agent.

132. The method of any one of claims 121-131, wherein the cancer is a solid tumor.

133. The method of any one of claims 121-131, wherein the cancer is bladder cancer, breast cancer, brain cancer, bone cancer, cervical cancer, colorectal cancer, head cancer, neck cancer, kidney cancer, liver cancer, lung cancer, lymphoma, mesothelioma, myeloma, prostate cancer, skin cancer, thyroid cancer, ovarian cancer, or uterine cancer.

134. The method of any one of claims 121-133, wherein the method elicits an anti-cancer immune response in the subject.

135. The method of any one of claims 121-134, wherein the method vaccinates the subject against the cancer.

136. A method of treating or preventing a viral infection in a subject in need thereof comprising administering a therapeutically effective amount of the nanoparticle of any one of claims 1-119 or a pharmaceutically acceptable salt thereof to the subject.

137. The method of claim 136, wherein the method treats the viral infection.

138. The method of claim 136, wherein the method prevents the viral infection.

139. The nanoparticle of claim 136-138, wherein the viral infection is hepatitis C, norovirus, junin, dengue virus, coronavirus, human immunodeficiency virus, herpes simplex, avian flu, chickenpox, cold sores, common cold, glandular fever, influenza, measles, mumps, pharyngitis, pneumonia, rubella, severe acute respiratory syndrome, and lower or upper respiratory tract infection (e.g., respiratory syncytial virus).

140. The nanoparticle of claim 139, wherein the viral infection is an influenza virus.

141. The nanoparticle of claim 139, wherein the viral infection is a human immunodeficiency virus.

142. The nanoparticle of claim 139, wherein the viral infection is a coronavirus.

143. The nanoparticle of claim 142, wherein the coronavirus is SARS-COV-2.

144. The nanoparticle of claim 143, wherein the SARS-COV-2 is the alpha, beta, gamma, delta, omicron strain, or BA.2 of SARS-COV-2.

145. The method of any one of claims 136-144, wherein the method elicits an immune response in the subject.

146. The method of any one of claims 136-145, wherein the method elicits an antiviral immune response in the subject.

147. The method of any one of claims 136-146, wherein the method vaccinates the subject against the viral infection.

148. A kit comprising chemotherapeutic agent and a nanoparticle of any one of claims 1-119.

149. A kit comprising chemotherapeutic agent and a pharmaceutical composition of claim 120.

150. The kit of claim 148 or 149, wherein the chemotherapeutic agent is cytotoxic.

151. The kit of any one of claims 148-150, wherein the chemotherapeutic agent is an alkylating agent, an antimetabolite, an anti-microtubule agent, anti-tumor antibiotic, or a topoisomerase inhibitor, a mitotic inhibitor, or a corticosteroid.

152. The kit of any one of claims 148-150, wherein the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat.

153. The kit of any one of claims 148-150, wherein the chemotherapeutic agent is doxorubicin.
